(19) United States
(12) Patent Application Publication
(10) Pub. No.: US 2009/0215857 A1
(43) Pub. Date: Aug. 27, 2009

(54) Therapeutic Pyrrolidines

(75) Inventors: Thomas Bruno Lanni, Macomb, MI (US); Scott Edward Lazerwith, San Francisco, CA (US); Susan Mary Kult Sheehan, Gales Ferry, CT (US); Anthony Jerome Thomas, Ann Arbor, MI (US)

Correspondence Address: PFIZER INC.;PATENT DEPARTMENT, Bld 114 M/S 114, EASTERN POINT ROAD, GROTON, CT, 06340, US

(73) Assignee: Pfizer Products Inc., Groto, CT (US)

(21) Appl. No.: 12/066,740

(22) PCT Filed: Aug. 29, 2006
(86) PCT No.: PCT/IB06/02457

§ 371 Date: Aug. 30, 2008

Related U.S. Application Data
(60) Provisional application No. 60/716,706, filed on Sep. 13, 2005.

(51) Int. Cl. A61K 31/40; C07D 207/36

(52) U.S. Cl. 514/424; 548/541

(57) ABSTRACT

The present invention provides for compounds of Formula (I),

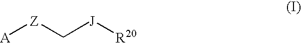

and pharmaceutically acceptable salts thereof, wherein A, J, Z, and R²⁰ have any of the values defined therefore in the specification, and pharmaceutically acceptable salts thereof, that are useful as agents in the treatment of disorders and conditions including attention deficit hyperactivity disorder, neuropathic pain, urinary incontinence, generalized anxiety disorder, depression, schizophrenia, and fibromyalgia. Also provided are pharmaceutical compositions comprising one or more compounds of Formula (I) or pharmaceutically acceptable salts thereof.

### BACKGROUND OF THE INVENTION

The monoamines norepinephrine and serotonin have a variety of effects as neurotransmitters. These monoamines are taken up by neurons after being released into the synaptic cleft. Norepinephrine and serotonin are taken up from the synaptic cleft by their respective norepinephrine and serotonin transporters.

Drugs that inhibit the norepinephrine and serotonin transporters can prolong the effects of norepinephrine and serotonin, respectively, in the synapse, providing treatment for a number of diseases. For example, the serotonin reuptake inhibitor fluoxetine has been found to be useful in the treatment of depression and other nervous system disorders. The norepinephrine reuptake inhibitor atomoxetine has been approved for the treatment of attention deficit hyperactivity disorder (ADHD) as STRATTERA®. In addition, the norepinephrine and serotonin transporter inhibitor milnacipran is being developed for the treatment of fibromyalgia, a disease that affects about 2% of the adult population in the United States. However, the FDA has not currently approved any drug for the treatment of fibromyalgia. Accordingly, there is an ongoing need in the art for compounds that are norepinephrine transporter inhibitors, serotonin transporter inhibitors, and that inhibit both norepinephrine and serotonin transporters, for the treatment of diseases including fibromyalgia, ADHD, neuropathic pain, urinary incontinence, generalized anxiety disorder, depression, schizophrenia.

### SUMMARY OF THE INVENTION

In one aspect, the present invention provides for compounds of formula I:

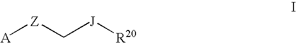

or a pharmaceutically acceptable salt thereof, wherein:
R²⁰ is a 3-pyrrolidinyl optionally substituted with one to eight substituents each independently selected from the group consisting of: C₁-C₄ alkyl, and halo; J is O or —N—R²², wherein R²² is H, C₁-C₄ alkyl, or —C(O)—C₁-C₄alkyl; Z is selected from the group consisting of: phenylene, naphthylene, a 5 to 6 membered heteroarylene, a 9 to 11-membered bicyclic arylene, and a 8 to 10-membered bicyclic heteroarylene, any of which may be optionally substituted with 1 to 5 substituents independently selected from the group consisting of: C₁-C₄ alkyl, C₁-C₄ alkoxy, halo, —CF₃, —CN, OH, C₁-C₄ alkyl-S—, and —NR³⁰R³¹, R³⁰ and R³¹ are each independently selected from the group consisting of: H, and C₁-C₄ alkyl; A is selected from the group consisting of: phenyl, naphthyl, a 5 to 6 membered heteroaryl, a 9 to 11-membered bicyclic aryl, and a 8 to 10-membered bicyclic heteroaryl, any of which may be optionally substituted with 1 to 5 substituents independently selected from the group consisting of: C₁-C₄ alkyl, C₁-C₄ alkoxy, halo, OH, —CN, —CF₃, CF₃O—, C₁-C₄ alkyl-S—, phenyl, C₁-C₄ alkyl-C(O)—, a 5 or 6 membered heteroaryl, a 5 to 7-membered heterocycloalkyl, C₁-C₄ alkyl-sulfonyl, —C(O)O—R¹², —C(O)NR¹⁴R¹⁶, —NR¹⁰R¹¹, —O-phenyl, and —O—CH₂-phenyl: R¹⁰ and R¹¹ are each independently selected from the group consisting of: H, —C(O)—C₁-C₄ alkyl, and C₁-C₄ alkyl; R¹² is H or C₁-C₄ alkyl; and R¹⁴ and R¹⁶ are each independently selected from the group consisting of: H and C₁-C₄ alkyl. In certain embodiments, Z is an optionally substituted phenylene with 1 to 5 substituents independently selected from the group consisting of: C₁-C₄ alkyl, C₁-C₄ alkoxy, halo, —CF₃, —CN, OH, C₁-C₄ alkyl-S—, and —NR³⁰R³¹, where R³⁰ and R³¹ are each independently selected from the group consisting of: H, and C₁-C₄ alkyl.

In certain embodiments, compounds of formula I, or a pharmaceutically acceptable salt thereof, that have the structure of formula II

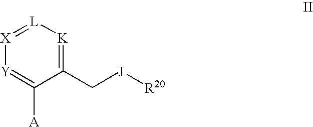

wherein: K is CR¹ or N; L is CR² or N; X is CR³ or N; Y is CR⁴ or N; where zero, one, or two of K, L, X and Y are N; R¹, R², R³, and R⁴ are each independently selected from the group consisting of: H, C₁-C₄ alkyl, C₁-C₄ alkoxy, halo, —CF₃, —CN, OH, C₁-C₄ alkyl-S—, and —NR³⁰R³¹, wherein R³⁰ and R³¹ are each independently selected from the group consisting of: H, and C₁-C₄ alkyl. In other embodiments, K is CR¹; L is CR²; X is CR³; and Y is CR⁴.

In certain embodiments, K is CR¹; L is CR²; X is CR³; and Y is N. In certain embodiments, K is N; L is CR²; X is CR³; and Y is CR⁴. In certain embodiments, K is CR¹; L is N; X is CR³; and Y is CR⁴. In certain embodiments, K is CR¹; L is CR²; X is N; and Y is CR⁴.

In other embodiments, A is

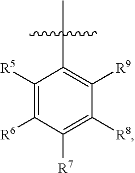

R⁵, R⁶, R⁷, R⁸, and R⁹ are each independently selected from the group consisting of: H, C₁-C₄ alkyl, C₁-C₄ alkoxy, halo, OH, —CN, —CF₃, CF₃O—, C₁-C₄ alkyl-S—, phenyl, C₁-C₄ alkyl-C(O)—, a 5 or 6 membered heteroaryl, a 5 to 7-membered heterocycloalkyl, C₁-C₄ alkyl-sulfonyl, —C(O)O—R¹², —C(O)NR¹⁴R¹⁶, NR¹⁰R¹¹, and —O—CH₂-phenyl, R¹⁰ and R¹¹ are each independently selected from the group consisting of: H, C(O)—C₁-C₄ alkyl, and C₁-C₄ alkyl; R¹² is H, or C₁-C₄ alkyl; and R¹⁴ and R¹⁶ are each independently selected from the group consisting of: H and C₁-C₄ alkyl.

The compounds of the present invention contain an unsubstituted or substituted 3-pyrrolidinyl group at R²⁰. The numbering of the ring atoms of an unsubstituted 3-pyrrolidinyl radical is provided below, where the number 3 designates the carbon of the 3-position of the 3-pyrrolidinyl radical:

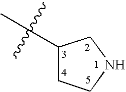

In certain embodiments, a compound of formula I may exist as a mixture of (R) and (S) stereoisomers at the 3-position of the optionally substituted 3-pyrrolidinyl ring. In other embodiments, a compound of formula I may exist as the (S) configuration at the 3-position of the optionally substituted 3-pyrrolidinyl ring. In other embodiments, a compound of formula I may exist as the (R) configuration at the 3-position of the optionally substituted 3-pyrrolidinyl ring.

In other embodiments are compounds of formula I, or a pharmaceutically acceptable salt thereof, J is O; K is CR¹; L is CR²; X is CR³; Y is CR⁴; and R²⁰ is an unsubstituted pyrrolidinyl. In other embodiments, R⁵, R⁶, R⁷, R⁸, and R⁹ are each independently selected from the group consisting of: H, C₁-C₄ alkyl, C₁-C₄ alkoxy, and halo. In still other embodiments, one or two of R⁵, R⁶, R⁷, R⁸, and R⁹ are each independently fluoro, methyl, or methoxy and three or four of R⁵, R⁶, R⁷, R⁸, and R⁹ are H. In yet other embodiments, R⁷ is fluoro, methyl, or methoxy and R⁵, R⁶, R⁸, and R⁹ are H; R⁷ and R⁹ are each independently fluoro, methyl, or methoxy and R⁵, R⁶, and R⁸ are H; or R⁸ and R⁹ are each independently fluoro, methyl, or methoxy, and R⁵, R⁶, and R⁷ are H. In yet other embodiments, one or two of R¹, R², R³, or R⁴ is halo and two to three of R¹, R², R³, or R⁴ are H. In other embodiments, one of R¹, R², R³, and R⁴ is halo and three of R¹, R², R³, and R⁴ are H. In certain embodiments, R², R³, or R⁴ is fluoro. In certain embodiments, R¹, R², R³, and R⁴ are H.

Examples of compounds of formula I include:
(S)-3-(4,2′,4′-trifluoro-biphenyl-2-ylmethoxy)-pyrrolidine;
(S)-3-(4,2′,3′-trifluoro-biphenyl-2-ylmethoxy)-pyrrolidine; and
(S)-3-(4,2′-difluoro-biphenyl-2-ylmethoxy)-pyrrolidine; and pharmaceutically acceptable salt thereof.

Examples of compounds of formula I, or a pharmaceutically acceptable salt thereof, also include:
(S)-3-(4′-methyl-biphenyl-2-ylmethoxy)-pyrrolidine;
(S)-3-(3′-4′-difluoro-biphenyl-2-ylmethyloxy)-pyrrolidine;
(S)-3-(2′,3′-difluoro-biphenyl-2-ylmethoxy)-pyrrolidine;
(S)-3-(biphenyl-2-ylmethoxy)-pyrrolidine; and
(S)-3-(4′-fluoro-biphenyl-2-ylmethoxy)-pyrrolidine; and pharmaceutically acceptable salt thereof.

Another example of a compound of formula I is (S)-3-(2′,4′-difluoro-biphenyl-2-ylmethoxy)-pyrrolidine, or a pharmaceutically acceptable salt thereof. In one particular embodiment, the compound is (S)-3-(2′,4′-difluoro-biphenyl-2-ylmethoxy)-pyrrolidine hydrochloride.

In other embodiments are compounds of formula I, or a pharmaceutically acceptable salt thereof, wherein said compound is (S)-3-(2′,4′-difluoro-biphenyl-2-ylmethoxy)-pyrrolidine hydrochloride, or said compound is selected from the group consisting of:
(S)-3-(4,2′,4′-trifluoro-biphenyl-2-ylmethoxy)-pyrrolidine;
(S)-3-(4,2′,3′-trifluoro-biphenyl-2-ylmethoxy)-pyrrolidine;
(S)-3-(4,2′-difluoro-biphenyl-2-ylmethoxy)-pyrrolidine;
(S)-3-(4′-methyl-biphenyl-2-ylmethoxy)-pyrrolidine;
(S)-3-(3′-4′-difluoro-biphenyl-2-ylmethyloxy)-pyrrolidine;
(S)-3-(2′,3′-difluoro-biphenyl-2-ylmethoxy)-pyrrolidine;
(S)-3-(biphenyl-2-ylmethoxy)-pyrrolidine;
(S)-3-(4′-fluoro-biphenyl-2-ylmethoxy)-pyrrolidine; and
(S)-3-(2′,4′-difluoro-biphenyl-2-ylmethoxy)-pyrrolidine;

or a pharmaceutically acceptable salt thereof.

In another aspect, the present invention provides for methods of treating a mammal suffering from a norepinephrine-mediated and/or serotonin-mediated disorder, the method comprising administering to a mammal in need of such treatment a therapeutically effective amount of a compound of formula I, or a pharmaceutically acceptable salt thereof.

In another aspect, the present invention provides for methods of treating attention deficit hyperactivity disorder (ADHD), the method comprising administering to a mammal in need of such treatment a therapeutically effective amount of a compound of formula I, or a pharmaceutically acceptable salt thereof.

In another aspect, the present invention provides for methods of treating a disease selected from the group consisting of: ADHD, neuropathic pain, urinary incontinence, generalized anxiety disorder, depression and schizophrenia, the method comprising administering to a mammal in need of such treatment a therapeutically effective amount of a compound of formula I, or a pharmaceutically acceptable salt thereof.

In another aspect, the present invention provides for methods of treating fibromyalgia, the method comprising administering to a mammal in need of such treatment a therapeutically effective amount of a compound of formula I, or a pharmaceutically acceptable salt thereof. In certain embodiments, the compound of formula I is (S)-3-(2′,4′-difluoro-biphenyl-2-ylmethoxy)-pyrrolidine, or a pharmaceutically acceptable salt thereof. In certain embodiments, the compound of formula I is (S)-3-(2′,4′-difluoro-biphenyl-2-ylmethoxy)-pyrrolidine hydrochloride.

In another aspect, the present invention provides for methods of treating a mammal suffering from a norepinephrine-mediated and/or serotonin-mediated disorder, the method comprising administering to a mammal in need of such treatment: (a) a compound of the formula I or a pharmaceutically acceptable salt thereof; (b) another pharmaceutically active compound that is an antidepressant or anti-anxiety agent, or a pharmaceutically acceptable salt thereof; and (c) a pharmaceutically acceptable excipient; wherein the active compounds “a” and “b” are present in amounts that render the composition effective in treating such disorder or condition.

In another aspect, the present invention provides for pharmaceutical compositions comprising: a therapeutically effective amount of a compound of formula I and a pharmaceutically acceptable excipient. In certain embodiments, the compound of formula I is (S)-3-(2′,4′-difluoro-biphenyl-2-ylmethoxy)-pyrrolidine, or a pharmaceutically acceptable salt thereof. In certain embodiments, the compound of formula I is (S)-3-(2′,4′-difluoro-biphenyl-2-ylmethoxy)-pyrrolidine hydrochloride.

### DETAILED DESCRIPTION OF THE INVENTION

The term “alkyl group” or “alkyl” means a monovalent radical of a straight or branched chain alkane. For example, a “C₁₋₄ alkyl” is an alkyl group having from 1 to 4 carbon atoms. Examples of C₁-C₄ straight-chain alkyl groups include methyl, ethyl, n-propyl, and n-butyl. Examples of branched-chain C₁-C₄ alkyl groups include isopropyl, tert-butyl, isobutyl, etc.

The term alkyl includes both “unsubstituted alkyl” and “substituted alkyl,” the latter of which refers to an alkyl group having one to six substituents replacing one to six hydrogen atoms, respectively, on the chain. The substituents may be on one or more carbons. Such substituents may be independently selected from the group consisting of: halo, —OH, —COOH, trifluoromethyl, —NH₂, —OCF₃, and —O—C₁-C₃ alkyl. In certain aspects of the present invention, halo is 1, Br, Cl, or F.

Typical substituted alkyl groups are 2-chloropropyl, 2-hydroxy-ethyl, 2-aminopropyl, trifluoromethyl, methoxyethyl, 1,2-dimethyl-propyl, pentachlorobutyl, and 4-chlorobutyl.

“C₁-C₄ alkoxy” refers to a straight chain or branched C₁₋₄ alkyl group bonded to an oxygen (i.e., —O—C₁-C₄ alkyl). Examples of C₁-C₄ alkoxy include methoxy, ethoxy, isopropoxy, tert-butoxy, and the like. The term “alkoxy” is intended to include both substituted and unsubstituted alkoxy groups. The term alkoxy includes both “unsubstituted alkoxys” and “substituted alkoxys,” the latter of which refers to —O-alkyl groups wherein the alkyl is substituted as described above.

Typical substituted alkoxy groups include aminomethoxy, trifluoromethoxy, 2-diethylaminoethoxy, 2-ethoxycarbonylethoxy, 3-hydroxypropoxy, and the like.

“Halo” includes fluoro, chloro, bromo, and iodo.

A “5-membered heteroaryl” is a monovalent radical of a 5-membered, monocyclic, heteroaromatic ring having from 1 to 4 carbon atoms and from 1 to 4 heteroatoms selected from the group consisting of O; S; and N; wherein the maximum number of O is 1, the maximum number of S is 1, and the maximum number of N is 4 and wherein the ring has carbon atoms and 1 O; carbon atoms and 1 S; carbon atoms and 1 N; carbon atoms and 2 N; carbon atoms and 3 N; carbon atoms and 4 N; carbon atoms, 1 S, and 1 N; carbon atoms, 1 S, and 2 N; carbon atoms, 1 O, and 1 N; or carbon atoms, 1 O, and 2 N. Examples of 5-membered heteroaryls include furanyl, 2-furanyl, 3-furanyl, imidazolyl, isoxazolyl, isothiazolyl, oxadiazolyl, oxazolyl, pyrazolyl, pyrrolyl, 2- or 3-pyrrolyl, thienyl, 2-thienyl, 3-thienyl, tetrazolyl, thiazolyl, thiadiazolyl, and triazolyl.

A “6-membered heteroaryl” is a monovalent radical of a 6-membered, monocyclic, heteroaromatic ring having from 3 to 5 carbon atoms and from 1 to 3 N. Examples of 6-membered heteroaryls include pyrazinyl, triazinyl, pyridinyl, pyrimidinyl, pyridin-2-yl, pyridin-4-yl, pyrimidin-2-yl, pyridazin-4-yl, and pyrazin-2-yl.

A 5 or 6 membered heteroaryl may be optionally substituted with 1 to 5 substituents independently selected from the group consisting of: C₁-C₄ alkyl, C₁-C₄ alkoxy, halo, OH, —CN, —CF₃, CF₃O—, C₁-C₄ alkyl-S—, phenyl, C₁-C₄ alkyl-C(O)—, a 5 or 6 membered heteroaryl, a 5 to 7-membered heterocycloalkyl, C₁-C₄ alkyl-sulfonyl, —C(O)O—R¹², —C(O)NR¹⁴R¹⁶, —NR¹⁰R¹¹, —O-phenyl, and —O—CH₂-phenyl: R¹⁰ and R¹¹ are each independently selected from the group consisting of: H, —C(O)—C₁-C₄ alkyl, and C₁-C₄ alkyl; R¹² is H or C₁-C₄ alkyl; and R¹⁴ and R¹⁶ are each independently selected from the group consisting of: H and C₁-C₄ alkyl. A heteroaryl can also include ring systems substituted on ring carbons with one or more —OH functional groups (which may further tautomerize to give a ring C═O group). A heteroaryl can also be optionally substituted on a ring sulfur atom by 1 or 2 oxygen atoms to give S═O, or SO₂ groups, respectively.

A “5-membered heteroarylene” is a divalent radical of a 5-membered, monocyclic, aromatic ring having from 1 to 4 carbon atoms and from 1 to 4 heteroatoms selected from the group consisting of: O; S; and N; wherein the maximum number of O is 1, the maximum number of S is 1, and the maximum number of N is 4 and wherein the ring has carbon atoms and 1 O; carbon atoms and 1 S; carbon atoms and 1 N; carbon atoms and 2 N; carbon atoms and 3 N; carbon atoms and 4 N; carbon atoms, 1 S, and 1 N; carbon atoms, 1 S, and 2 N; carbon atoms, 1 O, and 1 N; or carbon atoms, 1 O, and 2 N. Examples of 5-membered heteroarylenes include furanylene, 2-furanylene, 3-furanylene, imidazolylene, isoxazolylene, isothiazolylene, oxadiazolylene, oxazolylene, pyrazolylene, pyrrolylene, 2- or 3-pyrrolylene, thienylene, 2-thienylene, 3-thienylene, tetrazolylene, thiazolylene, thiadiazolylene, and triazolylene.

A “6-membered heteroaryiene” is a divalent radical of a 6-membered, monocyclic, aromatic ring having from 3 to 5 carbon atoms and from 1 to 3 N. Examples of 6-membered heteroarylenes include pyrazinylene, triazinylene, pyridinylene, pyrimidinylene, pyridin-2-ylene, pyridin-4-ylene, pyrimidin-2-ylene, pyridazin-4-ylene, and pyrazin-2-ylene.

A 5 or 6 membered heteroarylene may be optionally substituted with 1 to 5 substituents independently selected from the group consisting of: C₁-C₄ alkyl, C₁-C₄ alkoxy, halo, —CF₃, —CN, OH, C₁-C₄ alkyl-S—, and —NR³⁰R³⁰R³¹ where R³⁰ and R³¹ are each independently selected from the group consisting of: H, and C₁-C₄ alkyl.

A heteroarylene can also include ring systems substituted on ring carbons with one or more —OH functional groups (which may further tautomerize to give a ring C═O group). A heteroarylene can also be substituted on a ring sulfur atom by 1 or 2 oxygen atoms to give S═O, or SO₂ groups, respectively.

The term “C₅-C₇cycloalkyl” refers to a monovalent radical of a monocyclic alkane containing from 5 to 7 carbons. Examples of “C₅-C₇cycloalkyls” include cyclopentyl, cyclohexyl, and cycloheptyl. A “C₅-C₇cycloalkyl” may be unsubstituted or substituted with 1 or 2 groups independently selected from —OH, C₁-C₄alkyl, and —O—C₁-C₄alkyl.

A “5-membered heterocycloalkyl” is a monovalent radical of a 5-membered, monocyclic heterocycloalkane ring having from 2 to 4 carbon atoms and from 1 to 3 heteroatoms selected from the group consisting of: O; S; and N; wherein the maximum number of O is 1, the maximum number of S is 1, and the maximum number of N is 2, and wherein the ring has carbon atoms and 1 O; carbon atoms and 1 S; carbon atoms and 1 N; carbon atoms and 2 N; carbon atoms, 1 S, and 1 N; carbon atoms, 1 S, and 2 N; carbon atoms, 1 O, and 1 N; or carbon atoms, 1 O, and 2 N. Examples of 5-membered heterocycloalkyls include tetrahydrofuranyl, tetrahydrothienyl, imidazolidinyl, oxazolidinyl, imidazolinyl, isoxazolidinyl, and pyrrolidinyl.

A “6-membered heterocycloalkyl” is a monovalent radical of a 6-membered, monocyclic heterocycloalkane having from 3 to 5 carbon atoms and from 1 to 3 heteroatoms selected from the group consisting of: O; S; and N; wherein the maximum number of O is 1, the maximum number of S is 1, and the maximum number of N is 2, and wherein the ring has carbon atoms and 1 O; carbon atoms and 1 S; carbon atoms and 1 N; carbon atoms and 2 N; carbon atoms, 1 S, and 1 N; carbon atoms, 1 S, and 2 N; carbon atoms, 1 O, and 1 N; or carbon atoms, 1 O, and 2 N. Examples of 6-membered heterocycloalkyls include tetrahydropyranyl, dioxanyl, 1,3-dioxolanyl, 1,4-dithianyl, hexahydropyrimidinyl, morpholinyl, piperazinyl, piperidinyl, pyrazolidinyl, pyrazolinyl, 1,2,3,6-tetrahydropyridinyl, tetrahydrothiopyranyl, 1,1-dioxo-hexahydro-1λ⁶-thiopyranyl, 1,1-dioxo-1λ⁶-thiomorpholinyl, thiomorpholinyl, thioxanyl, and 1,3,5-trithianyl.

A “7-membered heterocycloalkyl” is a monovalent radical of a 7-membered, monocyclic heterocycloalkane having from 5 or 6 carbon atoms and from 1 to 3 heteroatoms selected from the group consisting of: O; S; and N; wherein the maximum number of O is 1, the maximum number of S is 1, and the maximum number of N is 2, and wherein the ring has carbon atoms and 1 O; carbon atoms and 1 S; carbon atoms and 1 N; carbon atoms and 2 N; carbon atoms, 1 S, and 1 N; carbon atoms, 1 S, and 2 N; carbon atoms, 1 O, and 1 N; or carbon atoms, 1 O, and 2 N. Examples of 7 membered heterocycloalkyls include azopanyl, oxepanyl, and thiepanyl.

A “9 to 11-membered bicyclic aryl” is a monovalent radical of a 9 to 11 membered bicyclic aromatic ring formed by the fusion of a benzene group to:

(1) a C₅₋₇ cycloalkane. Examples of a monovalent radical of a benzene ring fused to a C₅₋₇ cycloalkane include indanyl, 1,2,3,4-tetrahydro-naphthalenyl, and 6,7,8,9-tetrahydro-5H-benzocycloheptenyl; or

(2) a 5 to 7-membered heterocycloalkane. Examples of a monovalent radical of a benzene ring fused to a 5 to 7-membered heterocycloalkane include 2,3-dihydro-benzofuran-5-yl, 2,3-dihydro-benzo[1,4]dioxin-6-yl, and 3,4-dihydro-2H-benzo[b][1,4]dioxepin-7-yl.

A “9 to 11-membered bicyclic aryl” may be optionally substituted with 1 to 5 substituents independently selected from the group consisting of: C₁-C₄ alkyl, C₁-C₄ alkoxy, halo, OH, —CN, —CF₃, CF₃O—, C₁-C₄ alkyl-S—, phenyl, C₁-C₄ alkyl-C(O)—, a 5 or 6 membered heteroaryl, a 5 to 7-membered heterocycloalkyl, C₁-C₄ alkyl-sulfonyl, —C(O)O—R¹², —C(O)NR¹⁴R¹⁶, —NR¹⁰R¹¹, —O-phenyl, and —O—CH₂-phenyl; R¹⁰ and R¹¹ are each independently selected from the group consisting of: H, —C(O)—C₁-C₄ alkyl, and C₁-C₄ alkyl; R¹² is H or C₁-C₄ alkyl; and R¹⁴ and R¹⁶ are each independently selected from the group consisting of: H and C₁-C₄ alkyl.

A 9 to 11-membered bicyclic arylene is a divalent radical of a 9 to 11-membered bicyclic aromatic ring. A 9 to 11-membered bicyclic arylene may be optionally substituted with 1 to 5 substituents independently selected from the group consisting of: C₁-C₄ alkyl, C₁-C₄ alkoxy, halo, —CF₃, —CN, OH, C₁-C₄ alkyl-S—, and —NR³⁰R³¹, where R³⁰ and R³¹ are each independently selected from the group consisting of: H, and C₁-C₄ alkyl.

An “8 to 10-membered bicyclic heteroaryl” is an 8 to 10-membered monovalent radical formed by the fusion of:

(1) a 5-membered heteroaromatic ring to the same type of 5-membered heteroaromatic ring (e.g., furo[3,2-b]furanyl) or to a different type of 5-membered heteroaromatic ring (e.g., 1H-pyrrolo[2,3-b]pyridinyl, etc.);

(2) a 6-membered heteroaromatic ring to the same type of 6-membered heteroaromatic ring (e.g., 1,7-naphthyridinyl) or to a different type of 6-membered heteroaromatic ring (e.g., naphthyridinyl, pteridinyl, phthalazinyl, etc.);

(3) a 6-membered heteroaromatic ring to a 5-membered heteroaromatic ring

(e.g., purinyl, etc.)

(4) a 5 or 6-membered heteroaromatic ring to a C₅₋₇ cycloalkane (e.g., tetrahydroquinolinyl, tetrahydroquinazolinyl, etc.);

(4) a 5 or 6-membered heteroaromatic ring to a 5- to 7-membered heterocycloalkyl (e.g. tetrahydronaphthyridinyl, etc.); or

(5) a 5 or 6-membered heteroaromatic ring to a benzene group. Examples of a monovalent radical of a 5- or 6-membered heteroaromatic ring fused to a benzene group include benzimidazolyl, benzofuranyl, benzofurazanyl, 2H-1-benzopyranyl, benzothiadiazinyl, benzothiazinyl, benzothiazolyl, benzothiophenyl, benzoxazolyl, cinnolinyl, furopyridinyl, indolinyl, indolizinyl, indolyl, or 2-, 3-, 4-, 5-, 6-, or 7-indolyl, 3H-indolyl, quinazolinyl, quinoxalinyl, isoindolyl, quinolinyl, and isoquinolinyl, wherein the fusion junctions are at adjacent ring atoms. The fusion junctions may be at nitrogen (e.g., indolizinyl) or carbon atoms in the 5- or 6-membered heteroaryl, wherein bonding to a nitrogen at a fusion junction may be —N< but not —N═.

A “8 to 10-membered bicyclic heteroaryl” may be optionally substituted with 1 to 5 substituents independently selected from the group consisting of: C₁-C₄ alkyl, C₁-C₄ alkoxy, halo, OH, —CN, —CF₃, CF₃O—, C₁-C₄ alkyl-S—, phenyl, C₁-C₄ alkyl-C(O)—, a 5 or 6 membered heteroaryl, a 5 to 7-membered heterocycloalkyl, C₁-C₄ alkyl-sulfonyl, —C(O)O—R¹², —C(O)NR¹⁴R¹⁶, —NR¹⁰R¹¹, —O-phenyl, and —O—CH₂-phenyl; R¹⁰ and R¹¹ are each independently selected from the group consisting of: H, —C(O)—C₁-C₄ alkyl, and C₁-C₄ alkyl; R¹² is H or C₁-C₄ alkyl; and R¹⁴ and R¹⁶ are each independently selected from the group consisting of: H and C₁-C₄ alkyl.

An 8 to 10-membered bicyclic heteroarylene is an 8 to 10-membered bicyclic heteroaryl group having an additional monovalent radical, i.e., a divalent radical. An 8 to 10-membered bicyclic heteroarylene may be optionally substituted with 1 to 5 substituents independently selected from the group consisting of: C₁-C₄ alkyl, C₁-C₄ alkoxy, halo, —CF₃, —CN, OH, C₁-C₄ alkyl-S—, and —NR³⁰R³¹, where R³⁰ and R³¹ are each independently selected from the group consisting of: H, and C₁-C₄ alkyl.

Phenyl refers to a monovalent radical of benzene. Phenyl groups, unless otherwise noted, may be optionally substituted with 1 to 5 substituents independently selected from the group consisting of: C₁-C₄ alkyl, C₁-C₄ alkoxy, halo, OH, —CN, —CF₃, CF₃O—, C₁-C₄ alkyl-S—, phenyl, C₁-C₄ alkyl-C(O)—, a 5 or 6 membered heteroaryl, a 5 to 7-membered heterocycloalkyl, C₁-C₄ alkyl-sulfonyl, —C(O)O—R², —C(O)NR¹⁴R¹⁶, —NR¹⁴R₁₆, R¹⁰R¹¹, —O-phenyl, and —O—CH₂-phenyl; R¹⁰ and R¹¹ are each independently selected from the group consisting of: H, —C(O)—C₁-C₄ alkyl, and C₁-C₄ alkyl; R¹² is H or C₁-C₄ alkyl; and R¹⁴ and R¹⁶ are each independently selected from the group consisting of: H and C₁-C₄ alkyl.

Phenylene refers to a divalent radical of benzene, and may be optionally substituted with 1 to 5 substituents independently selected from the group consisting of: C₁-C₄ alkyl, C₁-C₄ alkoxy, halo, —CF₃, —CN, OH, C₁-C₄ alkyl-S—, and —NR³⁰R³¹, where R³⁰ and R³¹ are each independently selected from the group consisting of: H, and C₁-C₄ alkyl.

Naphthyl refers to a monovalent radical of naphthalene. Naphthyl groups, unless otherwise noted, may be optionally substituted with 1 to 5 substituents independently selected from the group consisting of: C₁-C₄ alkyl, C₁-C₄ alkoxy, halo, OH, —CN, —CF₃, CF₃O—, C₁-C₄ alkyl-S—, phenyl, C₁-C₄ alkyl-C(O)—, a 5 or 6 membered heteroaryl, a 5 to 7-membered heterocycloalkyl, C₁-C₄ alkyl-sulfonyl, —C(O)O—R¹², —C(O)NR¹⁴R¹⁶, —NR¹⁰R¹¹, —O-phenyl, and —O—CH₂-phenyl; R¹⁰ and R¹¹ are each independently selected from the group consisting of: H, —C(O)—C₁-C₄ alkyl, and C₁-C₄ alkyl; R¹² is H or C₁-C₄ alkyl; and R¹⁴ and R¹⁶ are each independently selected from the group consisting of: H and C₁-C₄ alkyl.

Naphthylene refers to a divalent radical of naphthalene, and may be optionally substituted with 1 to 5 substituents independently selected from the group consisting of: C₁-C₄ alkyl, C₁-C₄ alkoxy, halo, —CF₃, —CN, OH, C₁-C₄ alkyl-S—, and —NR³⁰R³¹, where R³⁰ and R³¹ are each independently selected from the group consisting of: H, and C₁-C₄ alkyl.

### Preparation of Compounds

General synthetic schemes for preparing compounds of formula I are set forth below.

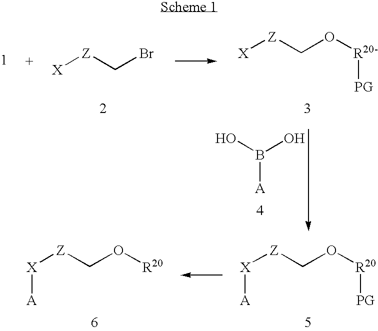

Scheme 1 depicts the synthesis of a pyrrolidinyl compound 6. An amino protected pyrrolidinol 1 (HO—R²⁰-PG) (N-(tert-butoxycarbonyl)-(S)-(+)-3-pyrrolidinol, N-(tert-butoxycarbonyl)-(R)-(−)-3-pyrrolidinol, etc.) may be treated with a hydride base (e.g., NaH) in a dry aprotic solvent (e.g., THF (tetrahydrofuran), DMF (dimethylformamide), DMSO (dimethylsulfoxide), toluene, CH₃CN, etc.). The reaction may be carried out at ambient temperature up to reflux temperature for about 30 minutes to an hour. The symbols A and

R²⁰ are those of formula I as set forth in the summary above, and Z is an optionally substituted phenylene group.

Then the reaction mixture may be contacted with 2 (e.g., 2-bromobenzyl bromide) to yield 3 (e.g., 3-(2-bromo-benzyloxy)-pyrrolidine-1-carboxylic acid tert-butyl ester). The reaction may be carried out at reflux for several hours. PG of 1 represents an amino protecting group. Those of skill in the art will recognize that a wide variety of protecting groups can be used as a suitable amino protecting group for PG of 1 (see e.g., Greene and Wuts, Protective Groups in Organic Synthesis, Wiley-Interscience; 3rd edition (1999). Suitable amino protecting groups include esters (tert-butyl ester (BOC), 9-fluorenylmethyl ester (Fmoc), benzyl ester, methyl ester, and allyl ester, etc.) and aryl sulfonyl derivatives (e.g., para-toluenesulfonyl, benzylsulfonyl, and phenylsulfonyl).

A solution of 3, where X is Br, may then be reacted with a boronic acid 4, such as a phenyl boronic acid (e.g., 2,4-difluorophenylboronic acid), followed by the addition of an inorganic carbonate base (e.g., Na₂CO₃, K₂CO₃, NaHCO₃, etc.) and a palladium catalyst such as tetrakis(triphenylphosphine)palladium(0) (Pd(PPh₃)₄), or Pd(Cl₂)dppf (dichloro (1,1 bis(diphenylphosphino) ferrocene) palladium(II)) to yield 5 (e.g., (S)-3-(2′,4′-difluoro-biphenyl-2-ylmethoxy)-pyrrolidine-1-carboxylic acid tert-butyl ester). The reaction may be carried out by refluxing for 2 to 4 hours or overnight in a suitable solvent such as THF or 1,2-dimethoxyethane. The corresponding potassium trifluoroborates, organoboranes, or boronate esters may be used in place of the boronic acid 4. The reaction of 3, where X is Cl, with 4 may be carried out using potassium fluoride, palladium acetate, and dicyclohexylphosphinobiphenyl in a solvent such as THF to provide 5.

The protecting group PG of 5 may then removed to provide a compound of formula 6 (e.g., (S)-3-(2′,4′-difluoro-biphenyl-2-ylmethoxy)-pyrrolidine). For example, a tert-butyl ester can be hydrolyzed from (S)-3-(2′,4′-difluoro-biphenyl-2-ylmethoxy)-pyrrolidine-1-carboxylic acid tert-butyl ester to provide (S)-3-(2′,4′-difluoro-biphenyl-2-ylmethoxy)-pyrrolidine using acids such as HCl or TFA (trifluoroacetic acid).

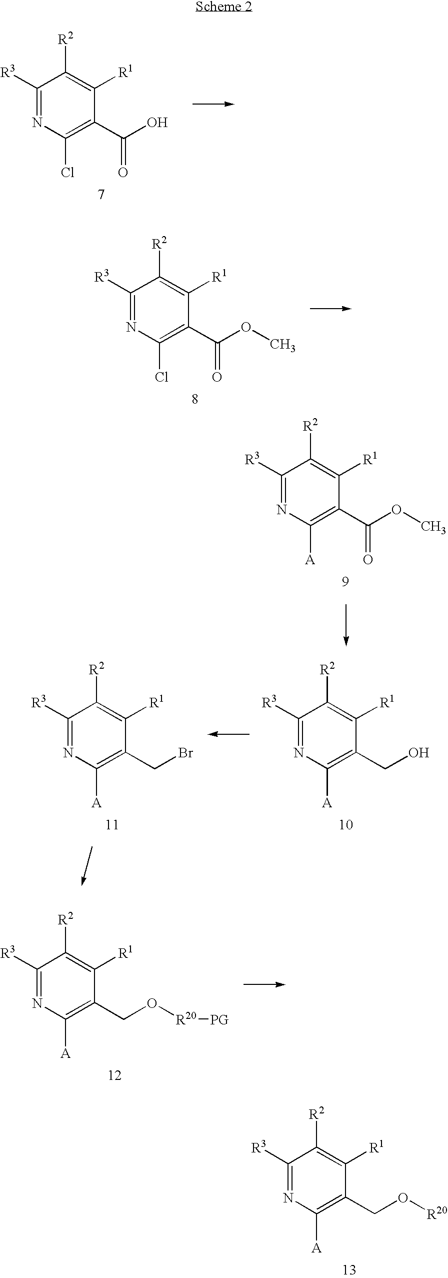

Scheme 2 depicts the synthesis of the pyrrolidinyl compound 13. A solution of 7 (e.g., 2-chloronicotinic acid) in 10 to 40% (v/v) MeOH in toluene may be treated with TMS diazomethane (trimethylsilyl diazomethane) to provide 8 (e.g., 2-chloronicotinic acid methyl ester). The reaction may be carried out at room temperature. The acid 7 may also be reacted with HCl (gas) in methanol to give 8. The symbols A, R¹, R², R³, and R²⁰ are those of formula II as set forth in the summary above.

The ester 8 may then be reacted with a boronic acid 4, such as a phenyl boronic acid (e.g., 2,4-difluorophenyl boronic acid), as described in Scheme 1 for the reaction of 3 to 5, to yield 9 (2-(2,4-difluoro-phenyl)-nicotinic acid methyl ester).

The compound 9 may then be reduced to yield 10 (e.g., [2-(2,4-difluoro-phenyl)-pyridin-3-yl]-methanol) using a reagent such as LiAlH₄ in dry THF or using NaBH₄ and CaCl₂ in an alcohol solvent such as ethanol.

The bromination of 10 using carbon tetrabromide in dry CH₂Cl₂ followed by the addition of triphenylphosphine yields 11 (e.g., 3-bromomethyl-2-(2,4-difluoro-phenyl)-pyridine). The reaction may be carried out at 0° C. to ambient temperature.

A solution of NaH treated 1 (see Scheme I) in dry THF or DMF (dimethylformamide) may then be reacted with 11 under reflux conditions to provide 12 (e.g., 3-[2-(2,4-difluoro-phenyl)-pyridin-3-ylmethoxy]-pyrrolidine-1-carboxylic acid tert-butyl ester). The protecting group PG of 12 may then removed (e.g., by hydrolysis with an acid such as HCl or TFA) provide a compound of formula 13 (e.g., (S)-3-(2,5-difluoro-phenyl)-2-(pyrrolidin-3-yloxymethyl)-pyridine).

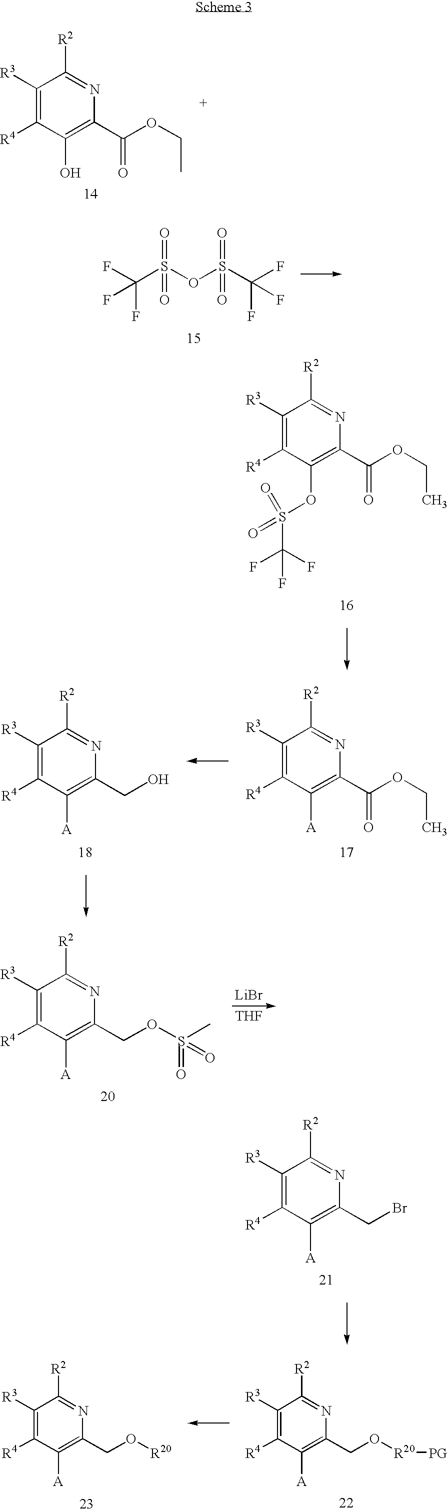

Scheme 3 depicts the synthesis of the pyrrolidinyl compound 23. The pyridine 14 (e.g., 3-hydroxy-pyridine-2-carboxylic acid ethyl ester) and an amine base (e.g., triethylamine) are reacted with trifluoromethanesulfonic anhydride (Tf₂O) to yield 16 (e.g., 3-trifluoromethanesulfonyloxy-pyridine-2-carboxylic acid ethyl ester). Typically the reaction can be carried out for 20 minutes to overnight at −10° C. to −30° C., in a solvent such as CHCl₃ or CH₂Cl₂. The symbols A, R², R³, R⁴, and R²⁰ are those of formula II as set forth in the summary above.

The ester 16 may then be reacted with an aryl boronic acid 4 (such as a phenyl boronic acid, e.g., 2,5-difluorophenyl boronic acid), as described in Scheme 1 for the reaction of 3 to 5, to yield 17 (e.g., 3-(2,5-difluoro-phenyl)-pyridine-2-carboxylic acid ethyl ester).

The compound 17 may then be reduced to the alcohol 18 (e.g., (e.g., [3-(2,5-difluoro-phenyl)-pyridin-2-yl]-methanol)) using a reagent such as LiAlH₄ in dry THF or using NaBH₄ and CaCl₂ in a suitable alcohol solvent such as ethanol, typically at room temperature to 60° C. for 30 minutes to overnight.

The alcohol 18 in a dry solvent such as dry CH₂Cl₂ may be reacted with an appropriate amine base (e.g., Et₃N or diisopropylamine) and a leaving group reagent 19 (e.g., methanesulfonyl chloride or toluenesulfonyl chloride) to provide 20 (e.g., methanesulfonic acid 3-(2,5-difluoro-phenyl)-pyridin-2-ylmethyl ester). The reaction may conveniently be carried out at about −40° C. for about 15 minutes. A solution of 20 in a suitable dry solvent such as dry THF may be reacted with LiBr, typically at 40 to 70° C. for one hour to overnight, to give 21 (e.g., 2-bromomethyl-3-(2,5-difluoro-phenyl)-pyridine).

A hydride base (e.g., NaH) treated 1 (see Scheme I) may be reacted with 21 in a dry solvent, such as dry THF or dry DMF, typically at reflux for 3 hours to overnight, to provide 22 (e.g., 3-[3-(2,5-difluoro-phenyl)-pyridin-2-ylmethoxy]-pyrrolidine-1-carboxylic acid tert-butyl ester). The protecting group PG of 22 may then be removed (e.g., via hydrolysis with acid such as HCl or TFA) to provide 23 (e.g., (S)-3-(2,5-difluoro-phenyl)-2-(pyrrolidin-3-yloxymethyl)-pyridine).

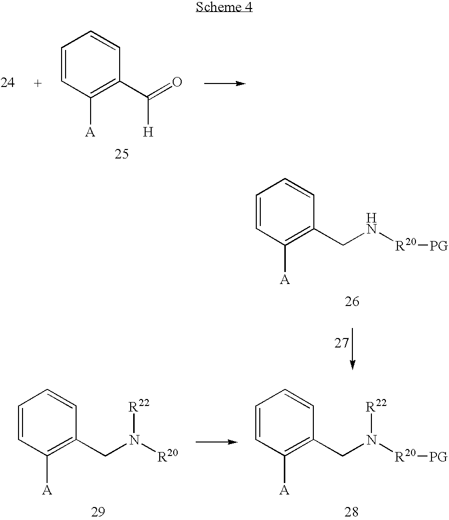

Scheme 4 depicts the synthesis of the pyrrolidinyl compound 29. An aldehyde 25 (e.g., biphenyl-2-carbaldehyde), is reductively amidated by reaction with an amino protected 3-amino-pyrrolidine 24 (e.g., (S)-3-amino-1-N-BOC-pyrrolidine), a reductant such as sodium triacetoxyborohydride, and MgSO₄ in a dry solvent such as dry CH₂Cl₂, dry 1,2-dichloroethane (DCE), or dry tetrahydrofuran to yield 26 (e.g., 3-[(biphenyl-2-ylmethyl)-amino]-pyrrolidine-1-carboxylic acid tert-butyl ester).

Those of skill in the art will recognize that a wide variety of protecting groups can be used as a suitable amino protecting group for PG of 24 (see e.g., Greene and Wuts, Protective Groups in Organic Synthesis, Wiley-Interscience; 3rd edition (1999). Suitable amino protecting groups include esters (tert-butyl ester (BOC), 9-fluorenylmethyl ester (Fmoc), benzyl ester, methyl ester, and allyl ester, etc.) and aryl sulfonyl derivatives (e.g., para-toluenesulfonyl, benzylsulfonyl, and phenylsulfonyl).

The compound 26 in a dry solvent (e.g., dry CH₃CN) is reacted with a carbonate base (e.g., Cs₂CO₃) followed by 27 (R²²—X, where X is Cl, Br, or I and R²² is a C₁-C₄ alkyl) (e.g., iodoethane) to provide 28 (e.g., 3-(biphenyl-2-ylmethyl-ethyl-amino)-pyrrolidine-1-carboxylic-acid tert-butyl ester). The protecting group PG of 28 may then be removed (e.g., via hydrolysis with acid such as HCl or TFA) to provide 29 (e.g., biphenyl-2-ylmethyl-ethyl-pyrrolidin-3-yl-amine).

### Pharmaceutically Acceptable Salts and Solvates

The compounds of formula I can exist in unsolvated forms as well as solvated forms, including hydrated forms. In general, the solvated forms, including hydrated forms are intended to be encompassed within the scope of the present invention. Some of the compounds of the present invention may exist as stereoisomers, including enantiomers, and diastereomers. Some compounds of the present invention have cycloalkyl groups, which may be substituted at more than one carbon atom, in which case all geometric forms thereof, both cis and trans and mixtures thereof, are within the scope of the present invention. All of these forms, including (R), (S), epimers, diastereomers, cis, trans, solvates (including hydrates), tautomers and mixtures thereof, are contemplated as compounds of the present invention.

Generally, compounds of the present invention will be administered as a formulation in association with one or more pharmaceutically acceptable excipients. The term “exciplent” is used herein to describe any ingredient other than the compound(s) of the invention. The choice of excipient will to a large extent depend on factors such as the particular mode of administration, the effect of the excipient on solubility and stability and the nature of the dosage form.

The compounds of the present invention (e.g., compounds of Formula I) may be capable of forming pharmaceutically acceptable salts, including but not limited to acid addition and/or base salts. Pharmaceutically acceptable salts of the compounds of formula (I) include the acid addition and base salts (including disalts) thereof. Examples of suitable salts can be found for example in Stahl and Wermuth, Handbook of Pharmaceutical Salts Properties, Selection, and Use, Wiley-VCH, Weinheim, Germany (2002); and Berge et al., “Pharmaceutical Salts,” J. of Pharmaceutical Science, 1977; 66:1-19.

Pharmaceutically acceptable acid addition salts of the compounds of Formula I include non-toxic salts derived from inorganic acids such as hydrochloric, nitric, phosphoric, sulfuric, hydrobromic, hydroiodic, phosphorus, and the like, as well as the salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include the acetate, aspartate, benzoate, besylate (benzenesulfonate), bicarbonate/carbonate, bisulfate, caprylate, camsylate (camphor sulfonate), chlorobenzoate, citrate, edisylate (1,2-ethane disulfonate), dihydrogenphosphate, dinitrobenzoate, esylate (ethane sulfonate), fumarate, gluceptate, gluconate, glucuronate, hibenzate, hydrochloride/chloride, hydrobromide/bromide, hydroiodide/iodide, isobutyrate, monohydrogen phosphate, isethionate, D-lactate, L-lactate, malate, maleate, malonate, mandelate, mesylate

(methanesulfonate), metaphosphate, methylbenzoate, methylsulfate, 2-napsylate (2-naphthalene sulfonate), nicotinate, nitrate, orotate, oxalate, palmoate, phenylacetate, phosphate, phthalate, propionate, pyrophosphate, pyrosulfate, saccharate, sebacate, stearate, suberate, succinate sulfate, sulfite, D-tartrate, L-tartrate, tosylate (toluene sulfonate), and xinafoate salts, and the like of compounds of Formula I. Also contemplated are the salts of amino acids such as arginate, gluconate, galacturonate, and the like.

Acid addition salts of the basic compounds may be prepared by contacting the free base form with a sufficient amount of the desired acid to produce a particular salt. The free base form may be regenerated by contacting the salt form with a base and isolating the free base. The free base forms may differ from their respective salt forms somewhat in certain physical properties such as solubility in polar solvents.

Pharmaceutically acceptable base addition salts may be formed with metals or amines, such as alkali and alkaline earth metal hydroxides, or of organic amines. Examples of metals used as cations are aluminum, calcium, magnesium, potassium, sodium, and the like. Examples of suitable amines include arginine, choline, chloroprocaine, N,N′-dibenzylethylenediamine, diethylamine, diethanolamine, diolamine, ethylenediamine (ethane-1,2-diamine), glycine, lysine, meglumine, N-methylglucamine, olamine, procaine (benzathine) and tromethamine.

The base addition salts of acidic compounds may be prepared by contacting the free acid form with a sufficient amount of the desired base to produce the salt in the conventional manner. The free acid form may be regenerated by contacting the salt form with an acid and isolating the free acid. The free acid forms may differ from their respective salt forms somewhat in certain physical properties such as solubility in polar solvents.

Pharmaceutical Compositions and Methods of Administration

This invention also provides for pharmaceutical compositions comprising a therapeutically effective amount of a compound of Formula I, or a pharmaceutically acceptable salt thereof together with a pharmaceutically acceptable excipient. The phrase “pharmaceutical composition” refers to a composition suitable for administration in medical or veterinary use. The phrase “therapeutically effective amount” means an amount of a compound, or a pharmaceutically acceptable salt thereof, sufficient to inhibit, halt, or allow an improvement in the disease being treated when administered alone or in conjunction with another pharmaceutical agent or treatment in a particular subject or subject population. For example in a human or other mammal, a therapeutically effective amount can be determined experimentally in a laboratory or clinical setting, for the particular disease and subject being treated.

It should be appreciated that determination of proper dosage forms, dosage amounts and routes of administration is within the level of ordinary skill in the pharmaceutical and medical arts and is described below.

A compound of the present invention can be formulated as a pharmaceutical composition in the form of a syrup, an elixir, a suspension, a powder, a granule, a tablet, a capsule, a lozenge, a troche, an aqueous solution, a cream, an ointment, a lotion, a gel, an emulsion, etc. Preferably, a compound of the present invention will cause a decrease in symptoms or disease indicia associated with a norepinephrine-mediated and/or serotonin-mediated disorder as measured quantitatively or qualitatively.

For preparing pharmaceutical compositions from the compounds of the present invention, pharmaceutically acceptable excipients can be either solid or liquid. Solid form preparations include powders, tablets, pills, capsules, cachets, suppositories and dispersible granules. A solid excipient can be one or more substances which may also act as diluents, flavoring agents, binders, preservatives, tablet disintegrating agents, or an encapsulating material.

In powders, the excipient is typically a finely divided solid which is in a mixture with the finely divided active component. In tablets, the active component is mixed with the excipient having the necessary binding properties in suitable proportions and compacted in the shape and size desired.

The powders and tablets contain from 1% to 95% (w/w) of the active compound. In certain embodiments, the active compound ranges from 5% to 70% (w/w). Suitable excipients are magnesium carbonate, magnesium stearate, talc, sugar, lactose, pectin, dextrin, starch, gelatin, tragacanth, methylcellulose, sodium carboxymethylcellulose, a low melting wax, cocoa butter and the like. The term “preparation” is intended to include the formulation of the active compound with encapsulating material as an excipient providing a capsule in which the active component with or without other excipients, is surrounded by a excipient, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture may then be poured into convenient sized molds, allowed to cool and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules and powders in vials or ampules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 1000 mg, preferably 1.0 mg to 100 mg, or from 1% to 95% (w/w) of a unit dose, according to the particular application and the potency of the active component. The composition can, if desired, also contain other compatible therapeutic agents.

Pharmaceutically acceptable excipients are determined in part by the particular composition being administered, as well as by the particular method used to administer the composition. Accordingly, there are a wide variety of suitable formulations of pharmaceutical compositions of the present invention (see, e.g., Remington: The Science and Practice of Pharmacy, 20th ed., Gennaro et al. Eds., Lippincott Williams and Wilkins, 2000).

A compound of the present invention, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane nitrogen and the like.

Formulations suitable for parenteral administration, such as, for example, by intravenous, intramuscular, intradermal and subcutaneous routes, include aqueous and non-aqueous, isotonic sterile injection solutions, which can contain antioxidants, buffers, bacteriostats and solutes that render the formulation isotonic with the blood of the intended recipient and aqueous and nonaqueous sterile suspensions that can include suspending agents, solubilizers, thickening agents, stabilizers and preservatives. In the practice of this invention, compositions can be administered, for example, by intravenous infusion, orally, topically, intraperitoneally, intravesically or intrathecally. The formulations of compounds can be presented in unit-dose or multi-dose sealed containers, such as ampules and vials. Injection solutions and suspensions can be prepared from sterile powders, granules and tablets of the kind previously described.

Examples of a typical tablet, parenteral and patch formulation include the following:

TABLET FORMULATION EXAMPLE 1
Tablet Formulation

| Ingredient | Amount |
|---|---|
| A Compound of Formula I | 50 mg |
| Lactose | 80 mg |
| Cornstarch (for mix) | 10 mg |
| Cornstarch (for paste) | 8 mg |
| Magnesium Stearate (1%) | 2 mg |
| | 150 mg |

The compounds of the present invention (e.g., a compound of Formula I, or a pharmaceutically acceptable salt thereof) can be mixed with the lactose and cornstarch (for mix) and blended to uniformity to a powder. The cornstarch (for paste) is suspended in 6 mL of water and heated with stirring to form a paste. The paste is added to the mixed powder and the mixture is granulated. The wet granules are passed through a No. 8 hard screen and dried at 50° C. The mixture is lubricated with 1% magnesium stearate and compressed into a tablet. The tablets are administered to a patient at the rate of 1 to 4 each day for treatment of a norepinephrine-mediated and/or serotonin-mediated disorder.

### Parenteral Solution Formulation Example 1

In a solution of 700 mL of propylene glycol and 200 mL of water for injection can be added 20.0 g of a compound of the present invention. The mixture is stirred and the pH is adjusted to 5.5 with hydrochloric acid. The volume is adjusted to 1000 mL with water for injection. The solution is sterilized, filled into 5.0 mL ampules, each containing 2.0 mL (40 mg of invention compound), and sealed under nitrogen. The solution is administered by injection to a subject suffering from a norepinephrine-mediated and/or serotonin-mediated disorder and in need of treatment.

### Patch Formulation Example 1

Ten milligrams of a compound of the present invention can be mixed with 1 mL of propylene glycol and 2 mg of acrylic-based polymer adhesive containing a resinous cross-linking agent. The mixture is applied to an impermeable backing (30 cm²) and applied to the upper back of a patient for sustained release treatment of a norepinephrine-mediated and/or serotonin-mediated disorder.

Methods for Treating Norepinephrine-Mediated and/or Serotonin-Mediated Disorders

The compounds of the present invention and pharmaceutical compositions comprising a compound of the present invention can be administered to treat a subject suffering from a norepinephrine-mediated and/or serotonin-mediated disorder, including central nervous disorders, which is alleviated by the inhibition of a norepinephrine transporters and/or serotonin transporters.

Norepinephrine-mediated and/or serotonin-mediated disorders can be treated prophylactically, acutely and chronically using compounds of the present invention, depending on the nature of the disease. Typically, the subject in each of these methods is human, although other mammals can also benefit from the administration of a compound of the present invention.

In therapeutic applications, the compounds of the present invention can be prepared and administered in a wide variety of oral and parenteral dosage forms. The term “administering” refers to the method of contacting a compound with a subject. Thus, the compounds of the present invention can be administered by injection, that is, intravenously, intramuscularly, intracutaneously, subcutaneously, intraduodenally, parentally, or intraperitoneally. Also, the compounds described herein can be administered by inhalation, for example, intranasally. Additionally, the compounds of the present invention can be administered transdermally, topically and via implantation. In certain embodiments, the compounds of the present invention are delivered orally. The compounds can also be delivered rectally, bucally, intravaginally, ocularly, or by insufflation.

The compounds utilized in the pharmaceutical method of the invention can be administered at a dosage of about 0.001 mg/kg to about 100 mg/kg daily. In certain embodiments, the daily dose range is from about 0.1 mg/kg to about 10 mg/kg.

The dose administered to a subject, in the context of the present invention should be sufficient to affect a beneficial therapeutic response in the subject over time. The term “subject” refers to a member of the class Mammalia. Examples of mammals include, without limitation, humans, primates, chimpanzees, rodents, mice, rats, rabbits, horses, livestock, dogs, cats, sheep and cows.

Determination of the proper dosage for a particular situation is within the skill of the practitioner. The dose will be determined by the efficacy of the particular compound employed and the condition of the subject, the severity of the disease being treated, as well as the body weight or surface area of the subject to be treated. The size of the dose also will be determined by the existence, nature and extent of any adverse side-effects that accompany the administration of a particular compound in a particular subject. In determining the effective amount of the compound to be administered in the treatment or prophylaxis of the disease being treated, the physician can evaluate factors such as the circulating plasma levels of the compound, compound toxicities, and/or the progression of the disease, etc. In addition, compounds of the present invention can be administered at a rate determined by factors that can include the pharmacokinetic profile of the compound, contraindicated drugs and the side-effects of the compound at various concentrations, as applied to the mass and overall health of the subject.

Generally, treatment is initiated with smaller dosages, which are less than the optimum dose of the compound. Thereafter, the dosage is increased by small increments until the optimum effect under circumstances is reached. For convenience, the total daily dosage may be divided and administered in portions during the day, if desired. The term “treatment” includes the acute, chronic, or prophylactic diminishment or alleviation of at least one symptom or characteristic associated with or caused by the disease being treated. For example, treatment can include diminishment of several symptoms of a disease, inhibition of the pathological progression of a disease, or complete eradication of a disease.

The present invention also relates to a method of treating a norepinephrine-mediated and/or serotonin-mediated disorder comprising administering to a mammal in need of such treatment a therapeutically effective amount of a compound of formula I. Examples of norepinephrine-mediated and/or serotonin-mediated disorders include fibromyalgia, single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; attention deficit hyperactivity disorder (ADHD); disruptive behavior disorder; behavioral disturbances associated with mental retardation, autistic disorder and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, palsys (e.g., Bell's palsy, cerebral palsy, birth palsy, brachial palsy, wasting palsy, ischemic palsy, progressive bulbar palsy and other palsys), and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremor; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy.

In one particular embodiment, patients suffering from fibromyalgia are administered a therapeutically effective amount of a compound of formula I, or a pharmaceutically acceptable salt thereof. Patients suffering from fibromyalgia typically exhibit a history of widespread pain, and the presence of pain at 11 out of 18 points upon palpatation (see e.g., Wolfe et al. (1990) Arthritis Rheum. 33:160-172). Fibromyalgia patients generally display pain perception abnormalities in the form of both allodynia (pain from innocuous stimulation) and hyperalgesia (an increased sensitivity to a painful stimulation).

Fibromyalgia patients typically also exhibit a range of other symptoms, including sleep disturbance and fatigue. Although less common than pain, fatigue, and sleep problems, a variety of other symptoms may occur as well. These include headaches, morning stiffness, difficulty concentrating, a circulatory problem that affects the small blood vessels of the skin (Raynaud's phenomenon), and irritable bowel syndrome. As with many conditions that cause chronic pain, anxiety and depression are common in fibromyalgia patients and may make symptoms worse. Symptoms may tend to come and go. There can be periods when the symptoms are constant (flares), which may be followed by periods when the symptoms are absent (remissions). Some fibromyalgia patients find that cold, damp weather, emotional stress, overexertion, and other factors exacerbate their symptoms.

A more specific embodiment of the present invention relates to the above method wherein the disorder or condition that is being treated is selected from major depression, single episode depression, recurrent depression, child abuse induced depression, postpartum depression, dysthymia, cyclothymia and bipolar disorder.

Another more specific embodiment of the present invention relates to the above method wherein the disorder or condition that is being treated is selected from schizophrenia, schizoaffective disorder, delusional disorder, substance-induced psychotic disorder, brief psychotic disorder, shared psychotic disorder, psychotic disorder due to a general medical condition, and schizophreniform disorder.

Another more specific embodiment of the present invention relates to the above method wherein the disorder or condition that is being treated is selected from autism, pervasive development disorder, and attention deficit hyperactivity disorder.

Another more specific embodiment of the present invention relates to the above method wherein the disorder or condition that is being treated is selected from generalized anxiety disorder, panic disorder, obsessive-compulsive disorder, post-traumatic stress disorder, and phobias, including social phobia, agoraphobia, and specific phobias.

Another more specific embodiment of the present invention relates to the above method wherein the disorder or condition that is being treated is selected from movement disorders such as akinesias, dyskinesias, including familial paroxysmal dyskinesias, spasticities, Tourette's syndrome, Scott syndrome, palsys (e.g., Bell's palsy, cerebral palsy, birth palsy, brachial palsy, wasting palsy, ischemic palsy, progressive bulbar palsy and other palsys), and akinetic-rigid syndrome; and extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremor.

Another more specific embodiment of the present invention relates to the above method wherein the disorder or condition that is being treated is pain. Pain refers to acute as well as chronic pain. Acute pain is usually short-lived and is associated with hyperactivity of the sympathetic nervous system. Examples are postoperative pain and allodynia. Chronic pain is usually defined as pain persisting from 3 to 6 months and includes somatogenic pain and psychogenic pain. Other pain is nociceptive.

Examples of the types of pain that can be treated with the compounds of formula I of the present invention and their pharmaceutically acceptable salts include pain resulting from soft tissue and peripheral damage, such as acute trauma, pain associated with osteoarthritis and rheumatoid arthritis, musculo-skeletal pain, such as pain experienced after trauma; spinal pain, dental pain, myofascial pain syndromes, epislotomy pain, and pain resulting from burns; deep and visceral pain, such as heart pain, muscle pain, eye pain, orofacial pain, for example, odontalgia, abdominal pain, gynaecological pain, for example, dysmenorrhoea, labor pain and pain associated with endometriosis; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, trigeminal neuralgia, neuropathic lower back pain, HIV related neuropathic pain, cancer related neuropathic pain, diabetic neuropathic pain, and arachnoiditis; neuropathic and non-neuropathic pain associated with carcinoma, often referred to as cancer pain; central nervous system pain, such as pain due to spinal cord or brain stem damage; lower back pain; sciatica; phantom limb pain, headache, including migraine and other vascular headaches, acute or chronic tension headache, cluster headache, temperomandibular pain and maxillary sinus pain; pain resulting from ankylosing spondylitis and gout; pain caused by increased bladder contractions; post operative pain; scar pain; and chronic non-neuropathic pain such as pain associated with fibromyalgia, HIV, rheumatoid and osteoarthritis, arthralgia and myalgia, sprains, strains and trauma such as broken bones; and post surgical pain.

Still other pain is caused by injury or infection of peripheral sensory nerves. It includes, but is not limited to pain from peripheral nerve trauma, herpes virus infection, diabetes mellitus, fibromyalgia, causalgia, plexus avulsion, neuroma, limb amputation, and vasculitis. Neuropathic pain is also caused by nerve damage from chronic alcoholism, human immunodeficiency virus infection, hypothyroidism, uremia, or vitamin deficiencies. Neuropathic pain includes, but is not limited to pain caused by nerve injury such as, for example, the diabetic neuropathic pain.

Psychogenic pain is that which occurs without an organic origin such as low back pain, atypical facial pain, and chronic headache.

Other types of pain are: inflammatory pain, osteoarthritic pain, trigeminal neuralgia, cancer pain, diabetic neuropathy, restless leg syndrome, acute herpetic and postherpetic neuralgia, causalgia, brachial plexus avulsion, occipital neuralgia, gout, phantom limb, burn, and other forms of neuralgia, neuropathic and idiopathic pain syndrome.

Another more specific embodiment of the present invention relates to the above method wherein the disorder or condition that is being treated is selected from delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies.

The compounds of the present invention can be co-administered to a subject. The term “co-administered” means the administration of two or more different pharmaceutical agents or treatments (e.g., radiation treatment) that are administered to a subject by combination in the same pharmaceutical composition or separate pharmaceutical compositions. Thus co-administration involves administration at the same time of a single pharmaceutical composition comprising two or more pharmaceutical agents or administration of two or more different compositions to the same subject at the same or different times. For example, a subject that is administered a first dosage that comprises a compound of the present invention at 8 a.m. and then is administered a second therapeutic agent at 1 to 12 hours later, e.g., 6 p.m., of that same day has been co-administered with a compound of the present invention and the second therapeutic agent. Alternatively, for example, a subject could be administered with a single dosage comprising a compound of the present invention and a second therapeutic agent at 8 a.m. has been co-administered with a compound of the present invention and the second therapeutic agent.

The compounds of the present invention may further be co-administered for the treatment of fibromyalgia with one or more agents useful for treating one or more indicia of fibromyalgia selected from the group consisting of: non-steroidal anti-inflammatory agents (hereinafter NSAIDs) such as piroxicam, loxoprofen, diclofenac, propionic acids such as naproxen, flurbiprofen, fenoprofen, ketoprofen and ibuprofen, ketorolac, nimesulide, acetominophen, fenamates such as mefenamic acid, indomethacin, sulindac, apazone, pyrazolones such as phenylbutazone, salicylates such as aspirin, COX-2 inhibitors such as CELEBREX® (celecoxib) and etoricoxib; steroids, cortisone, prednisone, muscle relaxants including cyclobenzaprine and tizanidine; hydrocodone, dextropropoxyphene, lidocaine, opioids, morphine, Fentanyl, tramadol, codeine, Paroxetine (PAXIL®), Diazepam, Femoxetine, Carbamazepine, Milnacipran (IXEL®), VESTRA®, Venlafaxine (EFFEXOR®), Duloxetine (CYMBALTA®), Topisetron (NAVOBAN®), Interferon alpha (Veldona), Cyclobenzaprine, CPE-215, Sodium oxbate (XYREM®), Celexa™ (citalopram HBr), ZOLOFT® (sertraline HCl), antidepressants, tricyclic antidepressants, Amitryptyline, Fluoxetine (PROZAC®), topiramate, escitalopram, benzodiazepenes including diazepam, bromazepam and tetrazepam, mianserin, clomipramine, imipramine, topiramate, and nortriptyline. The compound of the present invention may also be co-administered with alpha-2-delta ligands. Examples of alpha-2-delta ligands for use with the present invention are those compounds generally or specifically disclosed in U.S. Pat. No. 4,024,175, particularly gabapentin (NEURONTIN®), EP641330, particularly pregabalin (LYRICA®), U.S. Pat. No. 5,563,175, WO9733858, WO9733859, WO9931057, WO9931074, WO9729101, WO02085839, particularly [(1R,5R,6S)-6-(aminomethyl)bicyclo[3.2.0]hept-6-yl]acetic acid, WO9931075, particularly 3-(1-aminomethyl-cyclohexylmethyl)-4H-[1,2,4]oxadiazol-5-one and C-[1-(1H-tetrazol-5-ylmethyl)-cycloheptyl]-methylamine, WO9921824, particularly (3S,4S)-(1-aminomethyl-3,4-dimethyl-cyclopentyl)-acetic acid, WO0190052, Wo0128978, particularly (1α,3α,5α)(3-amino-methyl-bicyclo[3.2.0]hept-3-yl)-acetic acid, EP0641330, WO9817627, WO0076958, particularly (3S,5R)-3-aminomethyl-5-methyl-octanoic acid, POT/IB03/00976, particularly (3S,5R)-3-amino-5-methyl-heptanoic acid, (3S,5R)-3-amino-5-methyl-nonanoic acid and (3S,5R)-3-Amino-5-methyl-octanoic acid, EP1178034, EP1201240, WO9931074, WO03000642, WO0222568, WO0230871, WO0230881, WO02100392, WO02100347, WO0242414, WO0232736 and WO0228881, and pharmaceutically acceptable salts and solvates thereof, all of which are incorporated herein by reference.

For the treatment of depression, anxiety, schizophrenia, etc., the compounds of the present invention can be used in conjunction with one or more other antidepressants or anti-anxiety agents. Examples of classes of antidepressants that can be used in combination with the active compounds of the present invention include norepinephrine reuptake inhibitors, selective serotonin reuptake inhibitors (SRIs), NK-1 receptor antagonists, monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, alpha-2-delta ligands (A2D) (e.g., NEURONTIN®, and LYRICA®, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one and C-[1-(1H-tetrazol-5-ylmethyl)-cycloheptyl]-methylamine, (3S,4S)-(1-aminomethyl-3,4-dimethyl-cyclopentyl)-acetic acid, (1α,3α,5α)(3-amino-methyl-bicyclo[3.2.0]hept-3-yl)-acetic acid, (3S,5R)-3-aminomethyl-5-methyl-octanoic acid, (3S,5R)-3-amino-5-methyl-heptanoic acid, (3S,5R)-3-amino-5-methyl-nonanoic acid and (3S,5R)-3-amino-5-methyl-octanoic acid, etc.), and atypical antidepressants. Suitable norepinephrine reuptake inhibitors include tertiary amine tricyclics and secondary amine tricyclics. Suitable tertiary amine tricyclics and secondary amine tricyclics include amitriptyline, clomipramine, doxepin, imipramine, trimipramine, dothiepin, butripyline, iprindole, lofepramine, nortriptyline, protriptyline, amoxapine, desipramine and maprotiline. Suitable selective serotonin reuptake inhibitors include fluoxetine, fluvoxamine, paroxetine, citalopram, and sertraline. Examples of monoamine oxidase inhibitors include isocarboxazid, pheneizine, and tranylcyclopramine. Suitable reversible inhibitors of monoamine oxidase include moclobemide. Suitable serotonin and noradrenaline reuptake inhibitors for use in the present invention include venlafaxine and duloxetine. Suitable CRF antagonists include those compounds described in International Patent Application Nos. WO 94/13643, WO 94113644, WO 94/13661, WO 94/13676 and WO 94/13677. Suitable atypical anti-depressants include bupropion, lithium, nefazodone, trazodone and viloxazine. Suitable NK-1 receptor antagonists include those referred to in World Patent Publication WO 01/77100. Suitable A2D ligands include those referred to in World Patent Publications WO 99/21824, WO 01/90052, WO 01/28978, WO 98/17627, WO 00/76958, and WO 03/082807, and specifically NEURONTIN® and LYRICA®.

Suitable classes of anti-anxiety agents that can be used in combination with the active compounds of the present invention include benzodiazepines and serotonin IA (5-HT_{IA}) agonists or antagonists, especially 5-HT_{IA} partial agonists, and corticotropin releasing factor (CRF) antagonists. Suitable benzodiazepines include alprazolam, chlordiazepoxide, clonazepam, chlorazepate, diazepam, halazepam, lorazepam, oxazepam, and prazepam. Suitable 5-HT_{IA} receptor agonists or antagonists include buspirone, flesinoxan, gepirone and ipsapirone.

Suitable antipsychotic agents include both conventional and atypical antipsychotics.

Conventional antipsychotics are antagonists of dopamine (D₂) receptors. The atypical antipsychotics also have D₂ antagonistic properties but possess different binding kinetics to these receptors and activity at other receptors, particularly 5-HT_{2A}, 5-HT_{2C} and 5-HT_{2D} (Schmidt B et a, Soc. Neurosci. Abstr. 24:2177, 1998).

The class of atypical antipsychotics includes clozapine (CLOZARIL®), 8-chloro-11-(4-methyl-1-piperazinyl)-5H-dibenzo[b,e][1,4]diazepine (U.S. Pat. No. 3,539,573); risperidone (RISPERDAL®), 3-[2-[4-(6-fluoro-1,2-benzisoxazol-3-yl)piperidino]ethyl]-2-methyl-6,7,8,9-tetrahydro-4H-pyrido-[1,2-a]pyrimidin-4-one (U.S. Pat. No. 4,804,663); olanzapine (ZYPREXA®), 2-methyl-4-(4-methyl-1-piperazinyl)-10H-thieno[2,3-b][1,5]benzodiazepine (U.S. Pat. No. 5,229,382); quetiapine (SEROQUEL®), 5-[2-(4-dibenzo[b,f][1,4]thiazepin-11-yl-1-piperazinyl)ethoxy]ethanol (U.S. Pat. No. 4,879,288); aripiprazole (ABILIFY®), 7-{4-[4-(2,3-dichlorophenyl)-1-piperazinyl]-butoxy}-3,4-dihydro carbostyril and 7-{4-[4-(2,3-dichlorophenyl)-1-piperazinyl]-butoxy}-3,4-dihydro-2(1H)-quinolinone (U.S. Pat. Nos. 4,734,416 and 5,006,528); sertindole, 1-[2-[4-[5-chloro-1-(4-fluorophenyl)-1H-indol-3-yl]-1-piperidinyl]ethyl]imidazolidin-2-one (U.S. Pat. No. 4,710,500); amisulpride (U.S. Pat. No. 4,410,822); and ziprasidone (GEODON®), 5-[2[4-(1,2-benzisothiazol-3-yl)piperazin-3-yl]ethyl]-6-chloroindolin-2-one hydrochloride hydrate (U.S. Pat. No. 4,831,031).

### EXAMPLES

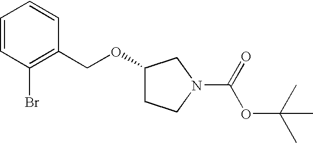

### Intermediate 1. (S)-3-(2-Bromo-benzyloxy)-pyrrolidine-1-carboxylic acid tert-butyl ester

To a room temperature stirred suspension of 95% NaH (0.961 g, 40.1 mmol) in dry THF (tetrahydrofuran) (227 mL) was added N-(tert-butoxycarbonyl)-(S)-(+)-3-pyrrolidinol (Sigma-Aldrich Corp., St. Louis, Mo., USA) (5.00 g, 26.7 mmol) in dry THF (20 mL) dropwise and the reaction was stirred for 40 minutes at room temperature. Then a solution of 2-bromobenzyl bromide (8.00, 32.1 mmol) in dry THF (20 mL) was added and the reaction was brought to reflux overnight with stirring. The heat was turned off and the reaction was allowed to cool to room temperature. The reaction was quenched with saturated NH₄C₁ and H₂O was added. The aqueous solution was extracted three times with 50 ml EtOAc (ethyl acetate) and the organic extracts were combined. The organic phase was dried over MgSO₄, filtered and concentrated down under reduced pressure. The residue was purified by silica chromatography using a gradient of hexanes:EtOAc (100:0) to hexanes:EtOAc (85:15) to yield 9.23 g (97.03%) of Intermediate 1 as a yellow oil.

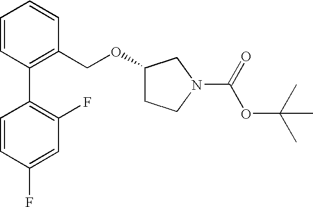

### Intermediate 2. (S)-3-(2′,4′-difluoro-biphenyl-2-ylmethoxy)-pyrrolidine-1-carboxylic acid tert-butyl ester

To a stirred solution of Intermediate 1 (2.00 g, 5.61 mmol) in dry THF (56 mL) was added the 2,4-difluorophenylboronic acid (1.77 g, 11.2 mmol) followed by Pd(PPh₃)₄ (tetrakis(triphenylphosphine)palladium(0)) (0.324 g, 0.281 mmol) and 2.0 N Na₂CO₃ (5.61 mL, 11.2 mmol). The mixture was then heated to reflux overnight with good stirring. The reaction was cooled to room temperature. The mixture was poured into H₂O (100 mL) and extracted four times with 25 ml CH₂Cl₂. The organic extracts were combined, washed twice with 30 ml brine, dried over Na₂SO₄, filtered and concentrated down under reduced pressure. The product was purified by silica chromatography using a gradient of hexanes:EtOAc (100:0) to hexanes:EtOAc (85:15) to yield 1.65 g (75.43%) of Intermediate 2 as a yellow oil.

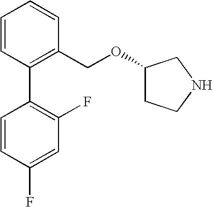

### Example A-1

### (S)-3-(2′,4′-Difluoro-biphenyl-2-ylmethoxy)-pyrrolidine fumarate

Step 1. To a room temperature stirred solution of Intermediate 2 (1.65 g, 4.23 mmol) in EtOAc (14.1 mL) was added an HCl solution (4.0 M in dioxane). The reaction was allowed to stir overnight at room temperature. The reaction was complete by HPLC (High Performance Liquid Chromatography) analysis and the reaction was concentrated under reduced pressure. EtOAc (20 mL) was added to the reaction and then concentrated down under reduced pressure. This process was repeated 3 times to provide (S)-3-(2′,4′-difluoro-biphenyl-2-ylmethoxy)-pyrrolidine hydrochloride.
Step 2. The free base of the hydrochloride salt was made by adding DOWEX 550 Anion (OH) Resin (Sigma-Aldrich Corp., St. Louis, Mo., USA) (5 g) to a stirred solution of the product in methanol (MeOH) (50 mL) for 30 minutes on a rotary evaporator. After 30 minutes, the mixture was filtered, the resin washed twice with 20 ml MeOH and concentrated down under reduced pressure to yield 1.209 g of free base.
Step 3. EtOAc (15 mL) was added to the free base and then fumaric acid (0.484 g, 1.0 eq) was added. The mixture was then concentrated down, a solution of EtOAc/Et₂O (2:1) was added, the mixture filtered and the product was washed three times with 15 ml diethyl ether (Et₂O). The white solid was placed in a drying oven at 60° C. under reduced pressure overnight to yield the title compound of Example A-1.

Examples A-3, A-5, A-6, A-9, A-10, A-13, A-15, A-16, A-19, A-20, A-22, and A-29 were synthesized in a manner similar to Example A-1 using the appropriate boronic acid and appropriate bromobenzyl bromide.

Example A-8 was synthesized in a manner similar to Example A-1, except maleic acid was substituted for fumaric acid to yield the particular maleate salt.

Examples A-2, A-4, A-7, A-11, A-12, A-14, A-17, A-18, A-21, A-27, and A-28 were synthesized in a manner similar to Example A-1, except only Step 1 was performed to yield the particular hydrochloride salt.

Examples A-30, A-31, and A-33 were synthesized in a manner similar to Example A-1, using the appropriate boronic acid and appropriate bromobenzyl bromide, and the synthesis of the intermediates that would correspond to Intermediate 1 were carried out using DMF instead of THF in a manner similar to that described below for Intermediate 1A.

Intermediate 1A. (S)-3-(2-Bromo-4-fluoro-benzyloxy)-pyrrolidine-1-carboxylic acid tert-butyl ester. To a room temperature stirred suspension of 95% NaH (0.257 g, 10.2 mmol) in dry DMF (dimethylformamide) (30 mL) was added N-(tert-butoxycarbonyl)-(S)-(+)-3-pyrrolidinol (Sigma-Aldrich Corp., St. Louis, Mo., USA) (1.30 g, 6.8 mmol) in dry DMF (14 mL) dropwise and the reaction was stirred for 1 hour at room temperature. A solution of 2-bromo-1-bromomethyl-4-fluoro-benzene (2.00 g, 7.50 mmol) in dry DMF (15 mL) was added and the reaction was continued at room temperature. After 4 hours, thin layer chromatography indicated that the starting material had been consumed. The reaction was quenched with saturated NH₄Cl and diluted with EtOAc (ethyl acetate). The aqueous layer was extracted three times with EtOAc (50 mL) and the organic extracts were combined. The organic layer was washed twice with H₂O, once with brine, dried over MgSO₄ and concentrated. The residue was purified by silica chromatography using 10 to 50% EtOAc/hexanes to yield 1.93 g (76%) of intermediate 1A as a clear, colorless oil.

Examples A-23, A-24, A-25, A-26, A-32, and A-34 were synthesized in a manner similar to Examples A-30 to A-33, except only Step 1 was performed to yield the particular hydrochloride salt. The names of the compounds of Examples A-2 to A-34 are set out in Table 1. The ¹H NMR and MS values for compounds of Examples A-1 to A-34 are set out in Table 2.

TABLE 1

| Ex. | STRUCTURE NAME |
|---|---|
| A-2 | (S)-3-(4′-Methyl-biphenyl-2-ylmethoxy)-pyrrolidine hydrochloride |
| A-3 | (S)-3-(4,2′,4′-Trifluoro-biphenyl-2-ylmethoxy)-pyrrolidine fumarate |
| A-4 | (S)-3-(4,2′,3′-Trifluoro-biphenyl-2-ylmethoxy)-pyrrolidine |
| | hydrochloride |
| A-5 | (S)-3-(3′-4′-Difluoro-biphenyl-2-ylmethyloxy)-pyrrolidine |
| | hydrochloride |
| A-6 | (S)-3-(2′,3′-Difluoro-biphenyl-2-ylmethoxy)-pyrrolidine fumarate |
| A-7 | (S)-3-(Biphenyl-2-ylmethoxy)-pyrrolidine hydrochloride |
| A-8 | (S)-3-(4′-Fluoro-biphenyl-2-ylmethoxy)-pyrrolidine maleate |
| A-9 | (S)-3-(4,2′-Difluoro-biphenyl-2-ylmethoxy)-pyrrolidine fumarate |
| A-10 | (S)-3-(4′-Methoxy-3′-methyl-biphenyl-2-ylmethoxy)-pyrrolidine |
| | fumarate |
| A-11 | (S)-3-(3′-Chloro-4′-fluoro-biphenyl-2-ylmethoxy)-pyrrolidine |
| | hydrochloride |
| A-12 | (S)-3-(4′-Fluoro-2′-methyl-biphenyl-2-ylmethoxy)-pyrrolidine |
| | hydrochloride |
| A-13 | (R)-3-(3′,4′-Difluoro-biphenyl-2-ylmethoxy)-pyrrolidine fumarate |
| A-14 | (R)-3-(Biphenyl-2-ylmethoxy)-pyrrolidine hydrochloride |
| A-15 | (R)-3-(2′,3′-Difluoro-biphenyl-2-ylmethoxy)-pyrrolidine fumarate |
| A-16 | (R)-3-(2′-Fluoro-biphenyl-2-ylmethoxy)-pyrrolidine fumarate |
| A-17 | (S)-3-(3′,5′-Difluoro-biphenyl-2-ylmethoxy)-pyrrolidine |
| | hydrochloride |
| A-18 | (S)-3-(3′-Trifluoromethyl-biphenyl-2-ylmethoxy)-pyrrolidine |
| | hydrochloride |
| A-19 | (S)-3-(2′-Isopropoxy-biphenyl-2-ylmethoxy)-pyrrolidine fumarate |
| A-20 | (R)-3-(2′,4′-Difluoro-biphenyl-2-ylmethoxy)-pyrrolidine fumarate |
| A-21 | (S)-3-(2′-Methylsulfanyl-biphenyl-2-ylmethoxy)-pyrrolidine |
| | hydrochloride |
| A-22 | (S)-3-(5′-Fluoro-2-methoxy-biphenyl-2-ylmethoxy)-pyrrolidine |
| | fumarate |
| A-23 | (S)-(5,2′,4′-Trifluoro-biphenyl-2-ylmethoxy)-pyrrolidine |
| | hydrochloride |
| A-24 | (S)-(5,2′,3′-Trifluoro-biphenyl-2-ylmethoxy)-pyrrolidine |
| | hydrochloride |
| A-25 | (S)-(5,2′-Difluoro-biphenyl-2-ylmethoxy)-pyrrolidine |
| | hydrochloride |
| A-26 | (S)-3-(6,2′,3′-Trifluoro-biphenyl-2-ylmethoxy)-pyrrolidine |
| | hydrochloride |
| A-27 | (S)-3-[2-(Pyrrolidin-3-yloxymethyl)-phenyl]-pyridine |
| | hydrochloride |
| A-28 | (S)-4-[2-(Pyrrolidin-3-yloxymethyl)-phenyl]-pyridine |
| | hydrochloride |
| A-29 | (S)-2-Methoxy-5-[2-(pyrrolidin-3-yloxymethyl)-phenyl]-pyridine |
| | fumarate |
| A-30 | (3S)-3-[(2′,3-difluorobiphenyl-2-yl)methoxy]pyrrolidine |
| | fumarate |
| A-31 | (3S)-3-[(2′,3′,4,5-tetrafluorobiphenyl-2-yl)methoxy]pyrrolidine |
| | fumarate |
| A-32 | (3S)-3-[(2′,6-difluorobiphenyl-2-yl)methoxy]pyrrolidine |
| | hydrochloride |
| A-33 | (3S)-3-[(2′,3,3′-trifluorobiphenyl-2-yl)methoxy]pyrrolidine |
| | fumarate |
| A-34 | (3S)-3-[(2′,4′,6-trifluorobiphenyl-2-yl)methoxy]pyrrolidine |
| | hydrochloride |

TABLE 2

| | | MS |
|---|---|---|
| Ex. | ¹H NMR | (M + 1) |
| A-1 | ¹H NMR (400 MHz, METHANOL-D4) δ ppm 1.9 (m, 1H) 2.0 (m, 1H) | 290 |
| | 3.2 (m, 2H) 3.2 (m, 2H) 3.3 (m, 2H) 4.1 (m, 1H) 4.4 (m, 2H) 7.1 (m, | |
| | 2H) 7.2 (d, J = 6.8 Hz, 1H) 7.3 (m, 1H) 7.4 (m, 2H) 7.5 (d, J = 7.2 Hz, 1H) | |
| A-2 | ¹H NMR (400 MHz, METHANOL-D4) δ ppm 1.9 (m, 1H) 2.1 (m, 1H) | 268 |
| | 2.4 (m, 3H) 3.2 (m, 1H) 3.2 (m, 1H) 3.3 (m, 4H) 4.2 (m, 1H) 4.5 (dd, | |
| | 2H) 7.2 (m, 5H) 7.3 (m, 2H) 7.5 (m, 1H) | |
| A-3 | ¹H NMR (400 MHz, METHANOL-D4) δ ppm 1.9 (m, 1H) 2.0 (m, 1H) | 308 |
| | 3.2 (dd, J = 12.6, 4.2 Hz, 1H) 3.3 (m, 4H) 4.1 (s, 1H) 4.3 (m, 2H) | |
| | 6.6 (s, 2H) 7.1 (m, 3H) 7.2 (m, J = 8.5, 5.8 Hz, 1H) 7.3 (m, 2H) | |
| A-4 | ¹H NMR (400 MHz, METHANOL-D4) δ ppm 1.9 (m, 1H) 2.0 (m, 1H) | 308 |
| | 3.2 (dd, J = 12.7, 4.3 Hz, 1H) 3.3 (m, 4H) 4.1 (t, J = 4.3 Hz, 1H) 4.4 (m, | |
| | 2H) 7.1 (m, 1H) 7.1 (m, 1H) 7.3 (m, 4H) | |
| A-5 | ¹H NMR (400 MHz, METHANOL-D4) δ ppm 2.0 (m, J = 9.5, 4.4 Hz, 1H) | 290 |
| | 2.1 (m, 1H) 3.2 (m, 1H) 3.3 (m, 3H) 4.2 (m, 1H) 4.4 (dd, 2H) | |
| | 7.2 (m, 1H) 7.3 (m, 3H) 7.4 (m, 2H) 7.5 (m, 1H) | |
| A-6 | ¹H NMR (400 MHz, METHANOL-D4) δ ppm 1.9 (m, 1H) 2.0 (m, 1H) | 290 |
| | 3.2 (m, 1H) 3.2 (m, 2H) 3.3 (m, 3H) 4.1 (m, 1H) 4.4 (d, J = 2.9 Hz, 2H) | |
| | 6.6 (s, 2H) 7.1 (m, 1H) 7.2 (m, 3H) 7.4 (m, 2H) 7.5 (d, J = 7.4 Hz, 1H) | |
| A-7 | ¹H NMR (400 MHz, DMSO-d6) δ ppm 1.79-1.90 (m, 2H) | 254 |
| | 3.03-3.12 (m, 3H) 3.16 (m, 1H) 4.06-4.14 (m, 1H) 4.28-4.39 (m, 2H) | |
| | 7.18-7.26 (m, 1H) 7.33-7.39 (m, 4H) 7.41-7.45 (m, 2H) 7.51 (m, 1H) | |
| | 9.19 (bs, 2H) | |
| A-8 | ¹H NMR (400 MHz, DMSO-D6) δ ppm 1.9 (m, 2H) 3.2 (m, 3H) | 272 |
| | 4.1 (m, J = 4.1 Hz, 1H) 4.3 (dd, 2H) 5.9 (s, 2H) 7.2 (m, 3H) 7.4 (m, 4H) | |
| | 7.5 (m, 1H) 8.8 (s, 2H) | |
| A-9 | ¹H NMR (400 MHz, METHANOL-D4) δ ppm 1.9 (m, 1H) 2.0 (m, 1H) | 290 |
| | 3.2 (dd, 1H) 3.2 (m, 2H) 3.3 (m, 2H) 4.1 (m, 1H) 4.4 (dd, J = 3.9 Hz, 2H) | |
| | 6.6 (s, 2H) 7.1 (m, 1H) 7.2 (m, 1H) 7.3 (m, 3H) 7.3 (m, 1H) | |
| | 7.4 (m, 1H) | |
| A-10 | ¹H NMR (400 MHz, METHANOL-D4) δ ppm 2.0 (m, 1H) 2.1 (m, 1H) | 298 |
| | 2.2 (s, 3H) 3.2 (dd, J = 12.6, 4.2 Hz, 1H) 3.3 (d, J = 10.1 Hz, 1H) | |
| | 3.3 (m, J = 2.4, 2.4 Hz, 2H) 3.9 (s, 3H) 4.2 (s, 1H) 4.5 (m, 2H) 6.6 (s, 2H) | |
| | 6.9 (d, J = 8.2 Hz, 1H) 7.1 (m, 2H) 7.2 (m, 1H) 7.3 (m, 2H) 7.5 (m, 1H) | |
| A-11 | ¹H NMR (400 MHz, METHANOL-D4) δ ppm 2.0 (m, 1H) 2.1 (m, 1H) | 306 |
| | 3.2 (dd, 1H) 3.3 (m, 5H) 4.2 (m, 1H) 4.4 (dd, 2H) 7.3 (m, J = 3.5 Hz, 1H) | |
| | 7.3 (d, J = 5.8 Hz, 2H) 7.4 (m, 2H) 7.5 (d, J = 7.2 Hz, 2H) | |
| A-12 | ¹H NMR (400 MHz, METHANOL-D4) δ ppm 1.9 (m, 2H) 2.0 (m, 3H) | 286 |
| | 3.2 (m, 4H) 4.0 (m, 1H) 4.2 (m, 2H) 4.3 (m, 1H) 7.0 (m, 1H) 7.0 (m, | |
| | 1H) 7.1 (t, J = 4.5 Hz, 2H) 7.4 (m, 2H) 7.5 (d, J = 1.8 Hz, 1H) | |
| A-13 | ¹H NMR (400 MHz, METHANOL-D4) δ ppm 2.0 (m, 1H) 2.1 (m, 1H) | 290 |
| | 3.2 (m, 1H) 3.3 (m, 3H) 3.3 (m, 1H) 4.2 (m, 1H) 4.4 (dd, 2H) 6.6 (s, | |
| | 2H) 7.2 (m, 1H) 7.3 (m, 1H) 7.3 (m, 2H) 7.4 (m, 2H) 7.5 (m, 1H) | |
| A-14 | ¹H NMR (400 MHz, DMSO-d6) δ ppm 1.79-1.90 (m, 2H) | 254 |
| | 3.03-3.12 (m, 3H) 3.16 (m, 1H) 4.06-4.14 (m, 1H) 4.28-4.39 (m, 2H) | |
| | 7.18-7.26 (m, 1H) 7.33-7.39 (m, 4H) 7.41-7.45 (m, 2H) 7.51 (m, 1H) | |
| | 9.19 (bs, 2H) | |
| A-15 | ¹H NMR (400 MHz, METHANOL-D4) δ ppm 1.9 (m, 1H) 2.0 (m, 1H) | 290 |
| | 3.1 (dd, J = 12.6, 4.2 Hz, 1H) 3.2 (m, 1H) 3.3 (m, 3H) 4.1 (t, J = 4.4 Hz, | |
| | 1H) 4.4 (m, 2H) 6.6 (s, 2H) 7.1 (dd, J = 7.8, 6.1 Hz, 1H) 7.2 (m, 2H) | |
| | 7.3 (d, J = 7.4 Hz, 1H) 7.4 (m, 2H) 7.5 (m, 1H) | |
| A-16 | ¹H NMR (400 MHz, METHANOL-D4) δ ppm 1.9 (m, 1H) 2.0 (m, 1H) | 272 |
| | 3.1 (dd, 1H) 3.2 (m, 2H) 3.3 (m, 2H) 4.4 (dd, 2H) 6.6 (s, 2H) 7.2 (m, | |
| | 1H) 7.2 (m, 2H) 7.3 (m, 1H) 7.4 (m, 4H) 7.5 (m, 1H) | |
| A-17 | ¹H NMR (400 MHz, METHANOL-D4) δ ppm 2.0 (m, 1H) 2.1 (m, 1H) | 290 |
| | 3.3 (m, 6H) 4.2 (m, 1H) 4.4 (m, 2H) 7.0 (m, 3H) 7.3 (m, 1H) 7.4 (m, | |
| | 2H) 7.5 (m, 1H) | |
| A-18 | ¹H NMR (400 MHz, DMSO-D6) δ ppm 1.9 (m, 2H) 3.1 (m, 1H) 3.1 (d, | 322 |
| | J = 4.1 Hz, 2H) 3.2 (m, 1H) 4.1 (m, 1H) 7.3 (dd, 2H) 7.4 (m, 2H) | |
| | 7.6 (m, 1H) 7.7 (m, 3H) 7.8 (m, 1H) 9.2 (m, J = 1.6 Hz, 2H) | |
| A-19 | ¹H NMR (400 MHz, METHANOL-D4) δ ppm 1.1 (d, J = 51.5 Hz, 6H) | 312 |
| | 1.9 (m, 1H) 2.0 (s, 1H) 3.1 (s, 2H) 3.3 (m, 2H) 4.1 (s, 1H) 4.4 (m, 3H) | |
| | 6.6 (s, 2H) 7.0 (m, 2H) 7.0 (m, 2H) 7.1 (m, 1H) 7.1 (m, 2H) | |
| | 7.3 (m, 3H) 7.5 (s, 1H) | |
| A-20 | ¹H NMR (400 MHz, METHANOL-D4) δ ppm 1.9 (m, 1H) 2.0 (m, 1H) | 290 |
| | 3.2 (m, 1H) 3.2 (m, 1H) 3.3 (m, 3H) 4.1 (m, 1H) 4.4 (dd, 2H) 6.6 (s, | |
| | 2H) 7.0 (d, J = 8.8 Hz, 2H) 7.2 (d, J = 7.0 Hz, 1H) 7.3 (d, J = 6.8 Hz, 1H) | |
| | 7.4 (dd, J = 7.1, 5.4 Hz, 2H) 7.5 (d, 1H) | |
| A-21 | ¹H NMR (400 MHz, METHANOL-D4) δ ppm 1.9 (m, 1H) 2.0 (m, 1H) | 300 |
| | 2.3 (m, 3H) 3.1 (m, 2H) 3.3 (m, 3H) 4.1 (m, 1H) 4.3 (m, 2H) 7.1 (m, | |
| | 2H) 7.2 (m, 1H) 7.4 (m, 4H) 7.5 (d, J = 7.2 Hz, 1H) | |
| A-22 | ¹H NMR (400 MHz, METHANOL-D4) δ ppm 1.9 (m, 2H) 3.1 (dd, | 302 |
| | J = 12.4, 3.8 Hz, 1H) 3.2 (s, 1H) 3.3 (m, 3H) 3.7 (s, 3H) 4.1 (s, 1H) | |
| | 4.3 (m, 2H) 6.6 (s, 2H) 6.9 (dd, J = 8.8, 2.9 Hz, 1H) 7.1 (m, 2H) | |
| | 7.1 (dd, J = 6.9, 1.9 Hz, 1H) 7.3 (m, 2H) 7.5 (m, 1H) | |
| A-23 | ¹H NMR (400 MHz, DMSO-d₆) δ ppm 1.70-1.92 (m, 2H), | 308 |
| | 2.91-3.08 (m, 1H), 3.07-3.11 (m, 2H), 3.12-3.24 (m, 1H), 3.97-4.09 (m, 1H), | |
| | 4.27 (s, 2H), 7.04-7.25 (m, 2H), 7.25-7.35 (m, 1H), | |
| | 7.34-7.50 (m, 2H), 7.54-7.66 (m, 1H), 9.25 (s, 2H) | |
| A-24 | ¹H NMR (400 MHz, DMSO-d₆) δ ppm 1.69-1.89 (m, 2H), | 308 |
| | 2.92-3.08 (m, 1H), 3.09 (d, J = 3.12 Hz, 2H), 3.12-3.23 (m, 1H), 3.89-4.09 (m, | |
| | 1H), 4.30 (s, 2H,) 7.13-7.25 (m, 2H), 7.26-7.37 (m, 2H,) | |
| | 7.45-7.58 (m, 1H), 7.58-7.67 (m, 1H), 9.21 (s, 2H) | |
| A-25 | ¹H NMR (400 MHz, DMSO-d₆) δ ppm 1.74-1.87 (m, 2H), | 290 |
| | 2.94-3.06 (m, 1H), 3.08 (d, J = 3.12 Hz, 2H), 3.11-3.21 (m, 1H), 3.97-4.06 (m, | |
| | 1H), 4.28 (s, 2H), 7.15 (dd, J = 9.55, 2.73 Hz, 1H), 7.20-7.34 (m, 3H), | |
| | 7.35-7.40 (m, 1H), 7.44-7.54 (m, 1H), 7.61 (dd, J = 8.58, 6.04 Hz, | |
| | 1H), 9.25 (s, 2H) | |
| A-26 | ¹H NMR (400 MHz, DMSO-d₆) δ ppm 1.65-1.95 (m, 2H), | 308 |
| | 2.95-3.09 (m, 1H), 3.08-3.13 (m, 2H), 3.13-3.24 (m, 1H), 3.91-4.09 (m, 1H), | |
| | 4.23-4.39 (m, 2H), 7.15-7.28 (m, 1H), 7.28-7.40 (m, 2H), | |
| | 7.44 (dd, J = 6.82, 2.92 Hz, 1H), 7.49-7.62 (m, 2H), 9.09 (s, 2H) | |
| A-27 | ¹H NMR (400 MHz, METHANOL-d4) δ ppm 1.99-2.11 (m, 1H) | 255 |
| | 2.13-2.22 (m, 1H) 3.27-3.32 (m, 3H) 3.33-3.40 (m, 2H) 4.30 (t, J = 4.39 Hz, | |
| | 1H) 4.49 (d, J = 1.75 Hz, 2H) 7.41-7.48 (m, 1H) 7.50-7.60 (m, 2H) | |
| | 7.61-7.66 (m, 1H) 8.20 (dd, J = 8.19, 5.85 Hz, 1H) 8.64-8.77 (m, | |
| | 1H) 8.92 (d, J = 5.65 Hz, 1H) 8.98 (d, J = 1.36 Hz, 1H) | |
| A-28 | ¹H NMR (400 MHz, METHANOL-d4) δ ppm 1.99-2.11 (m, 1H) | 255 |
| | 2.12-2.21 (m, 1H) 3.27-3.31 (m, 3H) 3.31-3.41 (m, 2H) 4.25-4.34 (m, | |
| | 1H) 4.49-4.62 (m, 2H) 7.44-7.53 (m, 1H) 7.53-7.63 (m, 2H) | |
| | 7.63-7.70 (m, 1H) 8.19 (d, J = 5.65 Hz, 2H) 8.94 (d, J = 5.65 Hz, 2H) | |
| A-29 | ¹H NMR (400 MHz, METHANOL-d4) δ ppm 1.92-2.05 (m, 1H) | 285 |
| | 2.08-2.18 (m, 1H) 3.17-3.27 (m, 1H) 3.27-3.38 (m, 3H) 3.95 (s, 3H) | |
| | 4.23 (s, 1H) 4.35-4.49 (m, 2H) 6.67 (s, 2H) 6.88 (d, J = 8.58 Hz, 1H) | |
| | 7.27 (dd, J = 5.07, 3.90 Hz, 1H) 7.33-7.44 (m, 2H) 7.48-7.54 (m, 1H) | |
| | 7.66-7.75 (m, 1H) 8.12 (d, J = 2.53 Hz, 1H) | |
| A-30 | ¹H NMR (400 MHz, DMSO-D6) δ ppm 1.7 (m, 2H) 2.9 (m, 2H) | 290 |
| | 3.1 (m, 2H) 3.9 (m, 1H) 4.3 (bs, 2H) 6.4 (s, 2H) 7.1 (m, 1H) 7.4 (m, 4H) | |
| | 7.5 (m, 2H) | |
| A-31 | ¹H NMR (400 MHz, DMSO-D6) δ ppm 1.8 (m, 2H) 3.1 (m, 4H) | 326 |
| | 4.1 (m, 1H) 4.3 (s, 2H) 6.4 (s, 2H) 7.2 (t, J = 7.1 Hz, 1H) 7.3 (m, 1H) | |
| | 7.5 (m, 2H) 7.7 (dd, J = 11.7, 8.4 Hz, 1H) | |
| A-32 | ¹H NMR (400 MHz, DMSO-D6) δ ppm 1.8 (m, 2H) 3.0 (m, 1H) | 290 |
| | 3.1 (m, 2H) 3.2 (m, 1H) 4.0 (m, 1H) 4.3 (m, 2H) 7.3 (m, 5H) 7.5 (m, 2H) | |
| | 9.2 (bs, 2H) | |
| A-33 | ¹H NMR (400 MHz, DMSO-D6) δ ppm 1.7 (m, 2H) 2.9 (m, 2H) | 308 |
| | 3.1 (m, 2H) 4.0 (m, 1H) 4.3 (bs, 2H) 6.4 (s, 2H) 7.2 (m, 2H) 7.3 (m, 2H) | |
| | 7.5 (m, 2H) | |
| A-34 | ¹H NMR (400 MHz, DMSO-D6) δ ppm 1.8 (m, 2H) 3.0 (m, 1H) | 308 |
| | 3.1 (s, 2H) 3.2 (m, 1H) 4.0 (m, 1H) 4.3 (m, 2H) 7.2 (t, J = 10.0 Hz, 1H) | |
| | 7.3 (t, J = 8.7 Hz, 1H) 7.5 (m, 4H) 9.1 (bs, 2H) | |

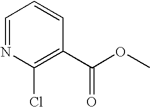

### Intermediate 3. 2-Chloronicotinic acid methyl ester

To a room temperature solution of 2-chloronicotinic acid (5.00 g, 31.7 mmol) in 20% MeOH/toluene (100 mL) was added TMS diazomethane (trimethylsilyl diazomethane) (2.0M in hexanes, 31.7 mL) dropwise and the reaction was monitored by TLC (thin-layer chromatography) until completion. The reaction was concentrated down under reduced pressure. The residue was purified by silica chromatography using a gradient of hexanes:EtOAc (100:0) to hexanes:EtOAc (80:20) to yield 4.65 g (85.42%) of Intermediate 3 as a yellow oil.

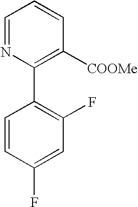

### Intermediate 4. 2-(2,4-Difluoro-phenyl)-nicotinic acid methyl ester

To a stirred solution of Intermediate 3 (1.00 g, 5.83 mmol) in THF (20 mL) was added the 2,4-difluorophenyl boronic acid (1.38 g, 8.74 mmol) followed by Pd(PPh₃)₄ (0.337 g, 0.291 mmol). The mixture was then heated to reflux for 1 hour and then the 2.0 N Na₂CO₃ (5.83 mL, 11.7 mmol) was added. The reaction was allowed to stir overnight at reflux. The suspension was cooled to room temperature. The mixture was poured into H₂O (50 mL) and extracted four times with 15 ml CH₂Cl₂. The organic extracts were combined, dried over Na₂SO₄, filtered and concentrated down under reduced pressure. The oil was purified by silica chromatography using a gradient of hexanes:EtOAc (100:0) to hexanes:EtOAc (75:25) to yield 1.35 g (92.71%) of Intermediate 4 as a yellow oil.

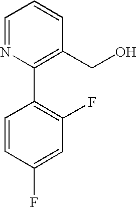

### Intermediate 5. [2-(2,4-Difluoro-phenyl)-pyridin-3-yl]-methanol

To a 0° C. stirred solution of Intermediate 4 (1.35 g, 5.40 mmol) in dry THF (100 mL) was added LiAlH₄ (lithium aluminum hydride) (1.0 M in THF, 10.8 mL) dropwise. The reaction was warmed to room temperature and monitored by HPLC until completion. Upon completion, it was quenched slowly with saturated NH₄Cl and the layers were separated. The aqueous layer was extracted three times with 15 ml EtOAc and the organic extracts were combined. The organic phase was dried over Na₂SO₄, filtered, and concentrated under reduced pressure. The product was purified by silica chromatography using a gradient of hexanes:EtOAc (100:0) to hexanes:EtOAc (60:40) to yield 0.408 g (34.15%) of Intermediate 5 as an oil.

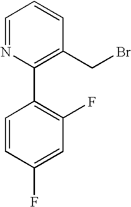

### Intermediate 6. 3-Bromomethyl-2-(2, difluoro-phenyl)-pyridine

To a 0° C. stirred solution of Intermediate 5 (0.408 g, 1.85 mmol) in dry CH₂Cl₂ (20 mL) was added carbon tetrabromide (0.734 g, 2.21 mmol) followed by the addition of triphenylphosphine (0.629, 2.40 mmol) portion wise. The reaction was warmed to room temperature overnight with good stirring. The mixture was filtered and the solid was washed twice with 15 ml CH₂Cl₂. The filtrate was concentrated down under reduced pressure and then purified by silica chromatography using a gradient of hexanes:EtOAc (100:0) to hexanes:EtOAc (85:15) to yield 0.203 g (38.70%) of Intermediate 6 an oil.

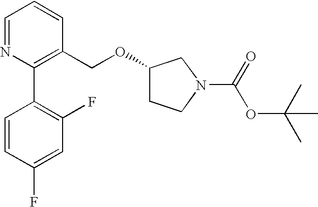

### Intermediate 7. (S)-3-[2-(2,4-Difluoro-phenyl)-pyridin-3-ylmethoxy]-pyrrolidine-1-carboxylic acid tert-butyl ester

To a room temperature stirred suspension of 95% NaH (0.021 g, 0.881 mmol) in dry THF (15 mL) was added N-(tert-butoxycarbonyl)-(S)-(+)-3-pyrrolidinol (0.110 g, 0.588 mmol) in dry THF (5 mL) dropwise and the reaction was stirred for 40 minutes at room temperature. A solution of Intermediate 6 (0.200 g, 0.705 mmol) dry THF (5 mL) was added and the reaction was heated to reflux overnight with good stirring. The reaction was cooled to room temperature. Saturated NH₄Cl was added to the reaction which was then extracted four times with 15 ml EtOAc. The organic extracts were combined, dried over MgSO₄, filtered and concentrated down under reduced pressure. The residue was purified by silica chromatography using a gradient of hexanes:EtOAc (100:0) to hexanes:EtOAc (85:15) to yield 0.108 g (46.94%) of Intermediate 7 as a yellow oil.

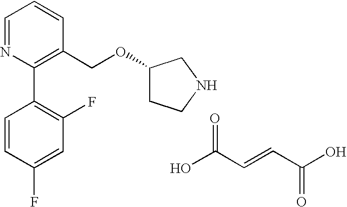

### Example B-1

### (S)-2-(2,4-Difluoro-phenyl)-3-(pyrrolidin-3-yloxymethyl)-pyridine fumarate

To a room temperature stirred solution of Intermediate 7 (0.107 g, 0.275 mmol) in EtOAc (1.86 mL) was added an HCl solution (4.0 M in Dioxane, 0.895 mL). The reaction was allowed to stir overnight at room temperature. The reaction was complete by HPLC, and was concentrated down under reduced pressure. EtOAc was added to the reaction and it was again concentrated under reduced pressure. DOWEX 550 Anion (OH) Resin was added to a stirred solution of the product in MeOH (5 mL) for 30 minutes made the free base of the salt. After 30 minutes, the mixture was filtered, the resin washed twice with 10 ml MeOH and the filtrate was concentrated under reduced pressure to yield 0.057 g of product. EtOAc (5 mL) was added to the free base and then fumaric acid (0.023 g, 1.0 eq) was added. The mixture was filtered, the solid was washed twice with 10 ml Et₂O and dried under reduced pressure in a drying oven at 60° C. to yield 0.081 g of the title compound as a white solid.

Examples B-2 and B-3 were synthesized in a manner similar to Example B-1, using the appropriate boronic acid and appropriate bromobenzyl bromide. The names of the compounds of Examples B-2 and B-3 are set out in Table 3. The ¹H NMR and MS values for compounds of Examples B-1 to B-3 are set out in Table 4.

TABLE 3

| Ex. | STRUCTURE NAME |
|---|---|
| B-2 | (S)-2-(2,3-Difluoro-phenyl)-3-(pyrrolidin-3-yloxymethyl)-pyridine |
| | fumarate |
| B-3 | (S)-2-(3,4-Difluoro-phenyl)-3-(pyrrolidin-3-yloxymethyl)-pyridine |
| | fumarate |

TABLE 4

| | ¹H NMR | MS |
|---|---|---|
| Ex. | ((400 MHz, METHANOL-d4) δ ppm) | (M + 1) |
| B-1 | 2.0 (m, 1H) 2.1 (m, 1H) 3.2 (m, 1H) 3.3 (m, 3H) 3.3 (m, 1H) 4.2 (m, 1H) | 291 |
| | 4.4 (dd, 2H) 6.6 (s, 2H) 7.1 (m, 2H) 7.5 (m, 1H) 7.5 (dd, J = 7.8, 5.1 Hz, | |
| | 1H) 8.0 (d, J = 8.0 Hz, 1H) 8.6 (d, J = 4.9 Hz, 1H) | |
| B-2 | 2.0 (m, 1H) 2.1 (m, 1H) 3.2 (m, 2H) 3.3 (m, 3H) 4.2 (s, 1H) 4.5 (s, 2H) | 291 |
| | 6.6 (s, 2H) 7.2 (m, 1H) 7.3 (m, 1H) 7.4 (m, 1H) 7.5 (m, 1H) 8.1 (d, | |
| | 1H) 8.6 (d, 1H) | |
| B-3 | 2.0 (m, 1H) 2.2 (m, 1H) 3.3 (m, 3H) 3.4 (m, 2H) 4.3 (m, 1H) 4.5 (dd, | 291 |
| | J = 6.0 Hz, 2H) 6.6 (s, 2H) 7.4 (m, 2H) 7.5 (m, J = 7.9, 4.8 Hz, 2H) | |
| | 8.0 (dd, J = 7.8, 1.8 Hz, 1H) 8.5 (d, J = 4.7 Hz, 1H) | |

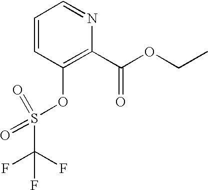

### Intermediate 8. 3-Trifluoromethanesulfonyloxy-pyridine-2-carboxylic acid ethyl ester

To a stirred solution of 3-hydroxy-pyridine-2-carboxylic acid ethyl ester (3.06 g, 20.0 mmol) and Et₃N (triethylamine) (5.58 mL, 40.0 mmol) in CH₂Cl₂ (100 mL) at minus 15° C. was added a solution of Tf₂O (trifluoromethanesulfonic anhydride) (4.04 mL, 24.0 mmol) in CH₂Cl₂ (10 mL). The reaction was stirred at this temperature for 1 hour and then CH₂Cl₂ (100 mL) was added followed by H₂O. The layers were separated and the organic layer was washed three times with 30 ml H₂O and twice with 30 ml brine. The organic phase was dried over Na₂SO₄, filtered and concentrated under reduced pressure. The residue was purified by silica chromatography using a gradient of hexanes:EtOAc (100:0) to hexanes:EtOAc (75:25) to yield 13.3 g (74.52%) of Intermediate 8 as a yellow oil.

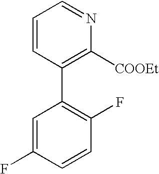

### Intermediate 9. 3-(2,5-Difluoro-phenyl)-pyridine-2-carboxylic acid ethyl ester

To a stirred solution of Intermediate 8 (1.58 g, 10.0 mmol) in dry THF (67 mL) was added the 2,5-difluorophenylboronic acid (2.00 g, 6.68 mmol) followed by Pd(PPh₃)₄ (0.386 g, 0.334 mmol) and 2.0 N Na₂CO₃ (6.68 mL, 13.4 mmol). The mixture heated at reflux overnight with good stirring. The suspension was cooled to room temperature. The mixture was poured into H₂O (100 mL) and extracted four times with 25 ml CH₂Cl₂. The organic extracts were combined, washed twice with 30 ml brine, dried over Na₂SO₄, filtered and concentrated under reduced pressure. The product was purified by silica chromatography using a gradient of hexanes:EtOAc (100:0) to hexanes:EtOAc (85:15) to yield 1.62 g (92.24%) of Intermediate 9 as a yellow solid.

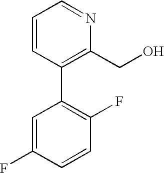

### Intermediate 10. [3-(2,5-Difluoro-phenyl)-pyridin-2-yl]-methanol

To a room temperature stirred solution of Intermediate 9 (1.23 g, 4.68 mmol) in dry EtOH (25 mL) was added in portions NaBH₄ (0.354 g, 9.35 mmol) followed by the addition of CaCl₂ (1.04 g, 9.35 mmol). reaction was heated at 60° C. overnight with good stirring. The reaction was complete by HPLC, therefore, the reaction was cooled to room temperature and concentrated under reduced pressure. H₂O (75 mL) was added to the solid and the aqueous layer was acidified to ˜pH 6 with 1N HCl. The aqueous layer was extracted five times with 20 ml EtOAc and the organic extracts were combined. The organic layer was dried over NaSO₄, filtered and concentrated down under reduced pressure to yield 1.36 g (100%) of Intermediate 10 as a yellow oil.

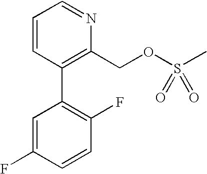

### Intermediate 11. Methanesulfonic acid 3-(2,5-difluoro-phenyl)-pyridin-2-ylmethyl ester

To a −40° C. stirred solution of Intermediate 10 (0.804 g, 3.63 mmol) in dry CH₂Cl₂ (13.5 mL) was added Et₃N (0.759 mL, 5.45 mmol) dropwise. The reaction was stirred for 15 minutes and methanesulfonyl chloride (0.337 mL, 4.36 mmol) was added. The reaction was stirred at the above temperature until reaction was complete. The reaction was complete by TLC, therefore, H₂O (100 mL) was added to the mixture and extracted three times with 20 ml CH₂Cl₂. The organic extracts were combined, dried over Na₂SO₄, filtered and concentrated down under reduced pressure to yield 0.405 g (100%) of Intermediate 10 as a yellow oil.

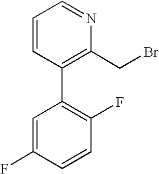

### Intermediate 12. 2-bromomethyl-3-(2,5-difluoro-phenyl)-pyridine

To a stirred solution of Intermediate 11 (1.09 g, 3.63 mmol) in dry THF (13.5 mL) was added LiBr (3.15 g, 36.3 mmol) and the reaction was heated at 50° C. The reaction was complete by HPLC, therefore, the reaction was cooled to room temperature and concentrated under reduced pressure. Saturated NH₄Cl (50 mL) was added to the residue and the aqueous layer was extracted four times with 20 ml CH₂Cl₂. The organic extracts were combined, dried over Na₂SO₄, filtered and concentrated down under reduced pressure to yield 0.385 g (100%) of Intermediate 12 as a yellow oil.

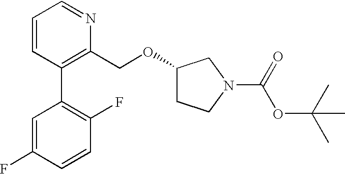

### Intermediate 13. (S)-3-[3-(2,5-difluoro-phenyl)-pyridin-2-ylmethoxy]-pyrrolidine-1-carboxylic acid tert-butyl ester

To a room temperature stirred suspension of 95% NaH (0.135 g, 5.61 mmol) in dry THF (28 mL) was added N-(tert-butoxycarbonyl)-(S)-(+)-3-pyrrolidinol (0.525 g, 2.80 mmol) in dry THF (5 mL) dropwise and the reaction was stirred for 40 minutes at room temperature. A solution of Intermediate 12 (0.956 g, 3.37 mmol) in dry THF (5 mL) was added and the reaction was brought to 85° C. overnight with good stirring. The reaction was complete by HPLC/MS, therefore, the reaction was cooled to room temperature. The reaction was quenched with saturated NH₄Cl and the aqueous layer was extracted four times with 20 ml EtOAc. The organic extracts were combined and washed twice with 10 ml brine. The organic phase was dried over Na₂SO₄, filtered and concentrated under reduced pressure. The reaction was purified by silica chromatography using a gradient of hexanes:EtOAc (100:0) to hexanes:EtOAc (70:30) to yield 0.296 g (67.76%) of Intermediate 13 as a yellow oil.

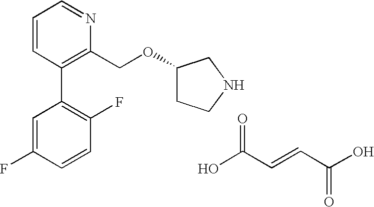

### Example C-1

### (S)-3-(2,5-Difluoro-phenyl)-2-(pyrrolidin-3-yloxymethyl)-pyridine fumarate

To a room temperature stirred solution of Intermediate 13 in dry EtOAc (2.53 mL) was added an HCl solution (4.0 M in dioxane). The reaction was stirred overnight at room temperature. The reaction was concentrated under reduced pressure, then EtOAc was added (3×25 mL) and concentrated again. The free base of the salt was made by adding DOWEX 550A (OH) resin (3 g) into a solution of the salt in MeOH (methanol) (20 mL). After 30 minutes of turning on a rotary evaporator without pressure, the resin was filtered and the filtrate was concentrated down under reduced pressure to yield 0.1869 g. Dry acetone (10 mL) was added to the free base, followed by fumaric acid (0.075 g, 1.0 eq). The mixture was concentrated down, a solution of EtOAc/Et₂O (2:1) was added, the mixture filtered, and the product was washed three times with 15 ml Et₂O (diethyl ether). The product was placed in a drying oven at 60° C. under reduced pressure overnight to yield 0.196 g of the title compound of Example C-1 as a white solid.

Example C-2 was synthesized in a manner similar to Example C-1, using the appropriate boronic acid and appropriate bromobenzyl bromide. The name of the compound of Example C-2 is set out in Table 5. The ¹H NMR and MS values for compounds of Examples C-1 and C-2 are set out in Table 6.

TABLE 5

| Ex. | STRUCTURE NAME |
|---|---|
| C-2 | (S)-3-(2,3-Difluoro-phenyl)-2-(pyrrolidin-3-yloxymethyl)-pyridine |
| | fumarate |

TABLE 6

| Ex. | ¹H NMR ((400 MHz, METHANOL-d4) δ ppm) | MS |
|---|---|---|
| C-1 | 2.0 (m, J = 9.7 Hz, 1H) 2.0 (m, 1H) 3.2 (d, J = 12.5 Hz, 1H) 3.3 (m, 4H) | 291 |
| | 4.2 (s, 1H) 4.5 (d, J = 2.7 Hz, 2H) 6.6 (s, 2H) 7.2 (m, 3H) 7.5 (m, 1H) | |
| | 7.8 (d, J = 7.8 Hz, 1H) 8.6 (d, J = 4.9 Hz, 1H) | |
| C-2 | 2.0 (m, J = 14.0, 9.6 Hz, 1H) 2.0 (m, 1H) 3.2 (dd, J = 12.7, 4.1 Hz, 1H) | 291 |
| | 3.3 (m, 4H) 4.2 (m, J = 4.3 Hz, 1H) 4.5 (d, J = 2.1 Hz, 2H) 6.6 (s, 2H) | |
| | 7.2 (m, J = 7.7, 6.1 Hz, 1H) 7.4 (m, J = 7.6 Hz, 1H) 7.5 (dd, J = 7.8, 4.9 Hz, | |
| | 1H) 7.8 (dd, J = 7.8, 1.6 Hz, 1H) 8.6 (d, J = 4.9, 1.6 Hz, 1H) | |

Examples D-1 to D-125 were synthesized in the following manner:

(S)-3-(2-bromo-5-fluoro-benzyloxy)-pyrrolidine-1-carboxylic acid tert-butyl ester (0.038 g, 0.102 mmol, an appropriately substituted boronic acid (0.134 mmol), Pd(PPh₃)₄ (0.0088 g, 0.0076 mmol) and 2.0N K₂CO₃ (0.042 g, 0.305 mmol) in dry DMF (0.067 M, 1.51 mL) were combined and placed into their reaction vessels for each different boronic acid. The closed vessels were allowed to agitate at 80° C. for 24 hours. The vessels were concentrated to dryness under reduced pressure and slurried with a solution of 10% MeOH/EtOAc (2 mL). The contents of each vessel was sonicated for 3-5 seconds and loaded onto a Bakerbound Silica tube (500 mg). Each vessel was rinsed with 10% MeOH/EtOAc (2 mL), poured onto the column and flushed with more 10% MeOH/EtOAc (2 mL). The vessels were concentrated under reduced pressure. 4N HCl-dioxane (4 mL) was added to each reaction vessel and they were shaken at room temperature overnight. The vessels were concentrated under reduced pressure. The crude products of Examples D-1 to D-116, and D-118 to D-125 were purified by preparative-scale HPLC (Phenomenex Fusion RP C18 100 mm×4.6 mm column (Phenomenex Inc., Torrance, Calif.), 5 to 98% acetonitrile+0.005% formic acid gradient in H₂O+0.005% formic acid over 4 minutes). For Example D-117, the purification was preparative-scale HPLC (Phenomenex Fusion RP C18 100 mm×4.6 mm column, 5 to 98% acetonitrile+0.005% NH₄OAc gradient in H₂O over 3 minutes). The names of the compounds of Examples D-2 to D-125 are set out in Table 7.

TABLE 7

| Ex. | STRUCTURE NAME |
|---|---|
| D-1 | (S)-3-(3′-Ethoxy-biphenyl-2-ylmethoxy)-pyrrolidine |
| D-2 | (S)-3-(4′-Methoxy-3′-methyl-biphenyl-2-ylmethoxy)-pyrrolidine |
| D-3 | (S)-3-(2′-Fluoro-biphenyl-2-ylmethoxy)-pyrrolidine |
| D-4 | (S)-3-(3′,4′-Dichloro-biphenyl-2-ylmethoxy)-pyrrolidine |
| D-5 | (S)-2′-(Pyrrolidin-3-yloxymethyl)-biphenyl-2-carboxylic acid diisopropylamide |
| D-6 | (S)-3-(3′-Fluoro-biphenyl-2-ylmethoxy)-pyrrolidine |
| D-7 | (S)-3-(3′,4′-Dimethyl-biphenyl-2-ylmethoxy)-pyrrolidine |
| D-8 | (S)-3-(2′,5′-Difluoro-biphenyl-2-ylmethoxy)-pyrrolidine |
| D-9 | (S)-3-(5′-Chloro-2′-fluoro-biphenyl-2-ylmethoxy)-pyrrolidine |
| D-10 | (S)-3-(2′-Fluoro-biphenyl-2-ylmethoxy)-pyrrolidine |
| D-11 | (S)-3-(4′-Fluoro-3′-methyl-biphenyl-2-ylmethoxy)-pyrrolidine |
| D-12 | (S)-3-(4′-Chloro-biphenyl-2-ylmethoxy)-pyrrolidine |
| D-13 | (S)-3-(3′-Chloro-biphenyl-2-ylmethoxy)-pyrrolidine |
| D-14 | (S)-3-(3′-Chloro-4′-fluoro-biphenyl-2-ylmethoxy)-pyrrolidine |
| D-15 | (S)-3-(2-Naphthalen-1-yl-benzyloxy)-pyrrolidine |
| D-16 | (S)-3-(3′-Methyl-biphenyl-2-ylmethoxy)-pyrrolidine |
| D-17 | (S)-3-(3′-Trifluoromethoxy-biphenyl-2-ylmethoxy)-pyrrolidine |
| D-18 | (S)-3-(2′-Chloro-5′-fluoro-biphenyl-2-ylmethoxy)-pyrrolidine |
| D-19 | (S)-3-(2′-Chloro-biphenyl-2-ylmethoxy)-pyrrolidine |
| D-20 | (S)-3-(3′-Methylsulfanyl-biphenyl-2-ylmethoxy)-pyrrolidine |
| D-21 | (S)-3-(3′-Fluoro-4′-methoxy-biphenyl-2-ylmethoxy)-pyrrolidine |
| D-22 | (S)-3-(3′-Methoxy-biphenyl-2-ylmethoxy)-pyrrolidine |
| D-23 | (S)-3-(2′,5′-Dimethyl-biphenyl-2-ylmethoxy)-pyrrolidine |
| D-24 | (S)-3-(2′-Methoxy-biphenyl-2-ylmethoxy)-pyrrolidine |
| D-25 | (S)-(4′-Methoxy-3′,5′-dimethyl-biphenyl-2-ylmethoxy)-pyrroldine |
| D-26 | (S)-3-(2′-Methyl-biphenyl-2-ylmethoxy)-pyrrolidine |
| D-27 | (S)-3-(3′,5′-Dimethyl-biphenyl-2-ylmethoxy)-pyrrolidine |
| D-28 | (S)-3-(4′-Methoxy-biphenyl-2-ylmethoxy)-pyrrolidine |
| D-29 | (S)-3-(2-Naphthalen-2-yl-benzyloxy)-pyrroldine |
| D-30 | (S)-2′-(Pyrrolidin-3-ylmethoxy)-biphenyl-3-carbonitrile |
| D-31 | (S)-3-(2′-Methoxy-biphenyl-2-ylmethoxy)-pyrrolidine |
| D-32 | (S)-3-[2-(2,3-Dihydro-benzofuran-5-yl)-benzyloxy]-pyrrolidine |
| D-33 | (S)-3-(2′,3′-Dimethyl-biphenyl-2-ylmethoxy)-pyrrolidine |
| D-34 | (S)-3-([1,1′,2′,1″]Terphenyl-2-ylmethoxy)-pyrrolidine |
| D-35 | (S)-3-(2′,3′-Dimethyl-biphenyl-2-ylmethoxy)-pyrrolidine |
| D-36 | (S)-3-(3′,4′-Dimethoxy-biphenyl-2-ylmethoxy)-pyrrolidine |
| D-37 | (S)-3-(2′-Trifluoromethoxy-biphenyl-2-ylmethoxy)-pyrrolidine |
| D-38 | (S)-[2′-(Pyrrolidin-3-yloxymethyl)-biphenyl-3-yl]-methanol |
| D-39 | (S)-(4′-Ethyl-biphenyl-2-ylmethoxy)-pyrrolidine |
| D-40 | (S)-3-(4′-Ethoxy-biphenyl-2-ylmethoxy)-pyrrolidine |
| D-41 | (S)-1-[2′-Pyrrolidin-3-yloxymethyl)-biphenyl-3-yl]-ethanone |
| D-42 | (S)-1-[2′-(Pyrrolidin-3-yloxymethyl)-biphenyl-2-yl]-ethanone |
| D-43 | (S)-3-(4′-Trifluoromethoxy-biphenyl-2-ylmethoxy)-pyrrolidine |
| D-44 | (S)-3-(5′-Chloro-2′-methoxy-biphenyl-2-ylmethoxy)-pyrrolidine |
| D-45 | (S)-1-[2′-(Pyrroldin-3-yloxymethyl)-biphenyl-3-yl]-1H-pyrazole |
| D-46 | (S)-3[2-(2,3-Dihydro-benzo[1,4]dioxin-6-yl-benzyloxy]-pyrrolidine |
| D-47 | (S)-1-[2′-(Pyrrolidin-3-yloxymethyl)-biphenyl-4-yl]-ethanone |
| D-48 | (S)-3-(2′-Benzyloxy-biphenyl-2-ylmethoxy)-pyrrolidine |
| D-49 | (S)-(5′-Isopropyl-2′-methoxy-biphenyl-2-ylmethoxy)-pyrrolidine |
| D-50 | (S)-3-[2-(3,4-Dihydro-2H-benzo[b][1,4]dioxepin-7-yl)-benzyloxy]-pyrrolidine |
| D-51 | (S)-2′-(Pyrrolidin-3-ylmethoxy)-biphenyl-4-carbonitrile |
| D-52 | (S)-2′-(Pyrrolidin-3-ylmethoxy)-biphenyl-2-carbonitrile |
| D-53 | (S)-2′-(Pyrrolidin-3-ylmethoxy)-biphenyl-3-carbonitrile |
| D-54 | (S)-3-(2′,4′-Dimethoxy-biphenyl-2-ylmethoxy)-pyrrolidine |
| D-55 | (S)-3-(4′-Trifluoromethyl-biphenyl-2-ylmethoxy)-pyrrolidine |
| D-56 | (S)-(4′-Isopropoxy-biphenyl-2-ylmethoxy)-pyrroldine |
| D-57 | (S)-3-(3′,4′,5′-Trimethoxy-biphenyl-2-ylmethoxy)-pyrrolidine |
| D-58 | (S)-3-(4′-Ethanesulfonyl-biphenyl-2-ylmethoxy)-pyrrolidine |
| D-59 | (S)-3,5-Dimethyl-1-[2′-(pyrrolidin-3-yloxymethyl)-biphenyl-3-yl]-1H-pyrazole |
| D-60 | (S)-3-Methoxy-2′-(pyrrolidin-3-yloxymethyl)-biphenyl-2-carboxylic acid |
| | diisopropylamide |
| D-61 | (S)-3-(5′-Chloro-4-fluoro-2′-methoxy-biphenyl-2-ylmethoxy)-pyrrolidine |
| D-62 | (S)-4′-Fluoro-2′-(pyrrolidin-3-yloxymethyl)-biphenyl-3-carbonitrile |
| D-63 | (S)-3-(4-Fluoro-4′-methyl-biphenyl-2-ylmethoxy)-pyrrolidine |
| D-64 | (S)-3-(4,5′-Difluoro-2′-methoxy-biphenyl-2-ylmethoxy)-pyrroldine |
| D-65 | (S)-3-(3′,5′-Dichloro-4-fluoro-biphenyl-2-ylmethoxy)-pyrrolidine |
| D-66 | (S)-3-(4-Fluoro-biphenyl-2-ylmethoxy)-pyrrolidine |
| D-67 | (S)-3-(4-Fluoro-3′,5′-dimethyl-biphenyl-2-ylmethoxy)-pyrrolidine |
| D-68 | (S)-3-(4-Fluoro-2′,5′-dimethoxy-biphenyl-2-ylmethoxy)-pyrrolidine |
| D-69 | (S)-3-(4-Fluoro-3′-methyl-biphenyl-2-ylmethoxy)-pyrrolidine |
| D-70 | (S)-3-(3′-Ethoxy-4-fluoro-biphenyl-2-ylmethoxy)-pyrrolidine |
| D-71 | (S)-(4,3′,4′-Trifluoro-biphenyl-2-ylmethoxy)-pyrrolidine |
| D-72 | (S)-3-(4-Fluoro-2′-methoxy-biphenyl-2-ylmethoxy)-pyrrolidine |
| D-73 | (S)-3-(3′-Chloro-4,4′-difluoro-biphenyl-2-ylmethoxy)-pyrrolidine |
| D-74 | (S)-3-(4,4′-Difluoro-biphenyl-2-ylmethoxy)-pyrrolidine |
| D-75 | (S)-3-(4-Fluoro-3′-trifluoromethyl-biphenyl-2-ylmethoxy)-pyrrolidine |
| D-76 | (S)-3-(4′-Ethyl-4-fluoro-biphenyl-2-ylmethoxy)-pyrrolidine |
| D-77 | (S)-3-(4-Fluoro-2′,4′-dimethoxy-biphenyl-2-ylmethoxy)-pyrrolidine |
| D-78 | (S)-1-[4′-Fluoro-2′-(pyrrolidin-3-yloxymethyl)-biphenyl-4-yl]-ethanone |
| D-79 | (S)-3-(4-Fluoro-4′-trifluoromethyl-biphenyl-2-ylmethoxy)-pyrrolidine |
| D-80 | (S)-3-(4,3′-Difluoro-biphenyl-2-ylmethoxy)-pyrrolidine |
| D-81 | (S)-1-[4′-Fluoro-2′-(pyrrolidin-3-yloxymethyl)-biphenyl-3-yl]-ethanone |
| D-82 | (S)-3-(4-Fluoro-2′,3′-dimethyl-biphenyl-2-ylmethoxy)-pyrrolidine |
| D-83 | (S)-3-(3′,4′-Dichloro-4-fluoro-biphenyl-2-ylmethoxy)-pyrrolidine |
| D-84 | (S)-3-(4-Fluoro-3′,4′-dimethoxy-biphenyl-2-ylmethoxy)-pyrrolidine |
| D-85 | (S)-3-(4′-Ethoxy-4-fluoro-biphenyl-2-ylmethoxy)-pyrrolidine |
| D-86 | (S)-(4,3′,5′-Trifluoro-biphenyl-2-ylmethoxy)-pyrrolidine |
| D-87 | (S)-[4′-Fluoro-2′-(pyrrolidin-3-yloxymethyl)-biphenyl-3-yl]-methanol |
| D-88 | (S)-4′-Fluoro-2′-(pyrrolidine-3-yloxymethyl)-biphenyl-4-carboxylic acid |
| | isopropyl ester |
| D-89 | (S)-3-(4-Fluoro-2′,5′-dimethyl-biphenyl-2-ylmethoxy)-pyrrolidine |
| D-90 | (S)-4′-Fluoro-2′-(pyrrolidin-3-yloxymethyl)-biphenyl-3-ol |
| D-91 | (S)-3-(4-Fluoro-4′-trifluoromethoxy-biphenyl-2-ylmethoxy)-pyrrolidine |
| D-92 | (S)-3-(4-Fluoro-4′-methoxy-3′,5′-dimethyl-biphenyl-2-ylmethoxy)-pyrrolidine |
| D-93 | (S)-3-(4,2′-Difluoro-3′-methoxy-biphenyl-2-ylmethoxy)-pyrrolidine |
| D-94 | (S)-3-(4,4′-Difluoro-3′-methyl-biphenyl-2-ylmethoxy)-pyrrolidine |
| D-95 | (S)-3-(4-Fluoro-3′-trifluoromethoxy-biphenyl-2-ylmethoxy)-pyrrolidine |
| D-96 | (S)-3-(4-Fluoro-2′-methoxy-5′-biphenyl-2-ylmethoxy)-pyrrolidine |
| D-97 | (S)-3-(2′-Benzyloxy-4-fluoro-biphenyl-2-ylmethoxy)-pyrrolidine |
| D-98 | (S)-3-(4-Fluoro-4′-methoxy-3′-methyl-biphenyl-2-ylmethoxy)-pyrrolidine |
| D-99 | (S)-4′-Fluoro-2′-(pyrrolidine-3-yloxymethyl)-biphenyl-4-ol |
| D-100 | (S)-3-(4,3′-Difluoro-4′-methoxy-biphenyl-2-ylmethoxy)-pyrrolidine |
| D-101 | (S)-3-(4″-Fluoro-[1,1′,2′,1″]terphenyl-2″-ylmethoxy)-pyrrolidine |
| D-102 | (S)-3-(4-Fluoro-4′-methanesulfonyl-biphenyl-2-methoxy)-pyrrolidine |
| D-103 | (S)-4′-Fluoro-2′-(pyrrolidin-3-yloxymethyl)-biphenyl-2-ol |
| D-104 | (S)-3-(4-Fluoro-3′,4′-dimethyl-biphenyl-2-ylmethoxy)-pyrrolidine |
| D-105 | (S)-3-(4-Fluoro-3′,4′,5′-trimethoxy-biphenyl-2-ylmethoxy-pyrrolidine |
| D-106 | (S)-3-(4,4′-Difluoro-2′-methyl-biphenyl-2-ylmethoxy)-pyrrolidine |
| D-107 | (S)-3-(4-Fluoro-4′-isopropoxy-biphenyl-2-ylmethoxy)-pyrrolidine |
| D-108 | (S)-3-(3′-Chloro-4-fluoro-biphenyl-2-ylmethoxy)-pyrrolidine |
| D-109 | (S)-3-(4′-Ethanesulfonyl-4-fluoro-biphenyl-2-ylmethoxy)-pyrrolidine |
| D-110 | (S)-4′-Fluoro-2′-(pyrrolidin-3-yloxymethyl)-biphenyl-4-carbonitrile |
| D-111 | (S)-3-(4′-Chloro-4-fluoro-biphenyl-2-ylmethoxy)-pyrrolidine |
| D-112 | (S)-1-[4′-Fluoro-2′-(pyrrolidin-3-yloxymethyl)-biphenyl-3-yl]-3,5-dimethyl-1H- |
| | pyrazole |
| D-113 | (S)-3-(2′-Ethyl-4-fluoro-biphenyl-2-ylmethoxy)-pyrrolidine |
| D-114 | (S)-3-(4-Fluoro-3′-methoxy-biphenyl-2-ylmethoxy)-pyrrolidine |
| D-115 | (S)-3-(4-Fluoro-4′-methoxy-biphenyl-2-ylmethoxy)-pyrrolidine |
| D-116 | (S)-1-[4′-Fluoro-2′-(pyrrolidin-3-yloxymethyl)-biphenyl-3-yl]-1H-pyrazole |
| D-117 | (S)-4′-Fluoro-2′-(pyrrolidin-3-yloxymethyl)-biphenyl-3-carboxylic acid amide |
| D-118 | (S)-2-Chloro-3-[2-(pyrrolidin-3-yloxymethyl)-phenyl]-pyridine |
| D-119 | (S)-2-Isopropoxy-3-[2-(pyrrolidin-3-yloxymethyl)-phenyl]-pyridine |
| D-120 | (S)-2-Ethoxy-3-[2-(pyrrolidin-3-yloxymethyl)-phenyl]-pyridine |
| D-121 | (S)-3-[2-(Pyrrolidin-3-yloxymethyl)-phenyl]-pyridin-2-ylamine |
| D-122 | (S)-5-[2-(Pyrrolidin-3-yloxymethyl)-phenyl]-quinoline |
| D-123 | (S)-5-[2-(Pyrrolidin-3-yloxymethyl)-phenyl]-isoquinoline |
| D-124 | (S)-5-[2-(Pyrrolidin-3-yloxymethyl)-phenyl]-1H-pyrrolo[2,3-b]pyridine |
| D-125 | (S)-1-{3-[2-Pyrrolidin-3-yloxymethyl)-phenyl]-pyridin-2-yl}-piperazine |

The MS, and retention time values were assayed for compounds of Examples D1 to D-125 on a Waters Alliance ZQ LC/MS system (Waters Corporation, Milford, Mass.). The system consists of a Alliance HT Waters 2795 Separation Module (Waters Corporation, Milford, Mass.) with a maximum capacity of 384 samples, a 2996 photo diode array detector, and a Micromass ZQ mass spectrometer (Waters Corporation, Milford, Mass.). The system is controlled through the Micromass MassLynx 4.0 software (Waters Corporation, Milford, Mass.). The ionization method used was atmospheric pressure chemical ionization (APCI), in positive and negative ion mode. The MS/APCI analyses were conducted at a probe temperature of 500□ C. and a cone voltage of 15V. The UV signal was recorded at 254 nm.

Separation was done by reversed-phase chromatography at pH 3.2, on a Phenomenex Fusion RP-C18; 100 mm×4.6 mm; 4 μm (Phenomenex Inc., Torrance, Calif.) with gradient elution and 0.005% formic acid added to the acetonitrile/water mobile phase. High aqueous/low organic solvent gradients consisting of 95-2% water, 5-98% acetonitrile, were used to perform the separations. The samples were eluted from the column over 8 minutes with a 2 minute column equilibration time (total run time was 10 minutes), at flow rate of 1 ml/min. The injected volume was 5 μl and samples were prepared in 900 μl CH₃CN:H₂O(1:1) by using a Gilson 215 sample prep system (Gilson, Inc., Middleton, Wis.), controlled through the 735 Gilson sampler software.

The solute retention time (t_{R}) is the time between the instant of sample introduction and when the detector senses the maximum of the retained peak. The time taken for an unretained solute to pass through the column and reach to the detector from the injection point is called the column dead time or holdup time (tₘ). The solute retention time is greater than the holdup time by the amount of time the solute spends in the stationary phase and is called the adjusted retention time (t_{R}′). These values lead to the fundamental relationship equation: t_{R}=t_{R}′+tₘ, describing retention in gas and liquid chromatography. Retention is usually measured in units of time for convenience. The MS and retention time values for compounds of Examples D-1 to D-125 are set out in Table 8:

TABLE 8

| | | RETENTION |
|---|---|---|
| | MS | TIME |
| Ex. | (M + 1) | (minutes) |
| D-1 | 298 | 2.51 |
| D-2 | 298 | 2.54 |
| D-3 | 272 | 2.36 |
| D-4 | 322 | 2.63 |
| D-5 | 381 | 2.53 |
| D-6 | 272 | 2.44 |
| D-7 | 282 | 2.58 |
| D-8 | 290 | 2.44 |
| D-9 | 306 | 2.49 |
| D-10 | 272 | 2.36 |
| D-11 | 286 | 2.55 |
| D-12 | 288 | 2.55 |
| D-13 | 288 | 2.52 |
| D-14 | 306 | 2.57 |
| D-15 | 304 | 2.60 |
| D-16 | 268 | 2.49 |
| D-17 | 338 | 2.63 |
| D-18 | 306 | 2.50 |
| D-19 | 288 | 2.44 |
| D-20 | 300 | 2.52 |
| D-21 | 302 | 2.46 |
| D-22 | 284 | 2.46 |
| D-23 | 282 | 2.55 |
| D-24 | 284 | 2.41 |
| D-25 | 312 | 2.52 |
| D-26 | 268 | 2.49 |
| D-27 | 282 | 2.61 |
| D-28 | 284 | 2.41 |
| D-29 | 304 | 2.61 |
| D-30 | 279 | 2.33 |
| D-31 | 284 | 2.38 |
| D-32 | 296 | 2.40 |
| D-33 | 282 | 2.57 |
| D-34 | 330 | 2.63 |
| D-35 | 282 | 2.57 |
| D-36 | 314 | 2.34 |
| D-37 | 338 | 2.60 |
| D-38 | 284 | 2.87 |
| D-39 | 282 | 2.59 |
| D-40 | 298 | 2.51 |
| D-41 | 296 | 2.34 |
| D-42 | 296 | 3.05 |
| D-43 | 338 | 2.66 |
| D-44 | 318 | 2.49 |
| D-45 | 320 | 2.38 |
| D-46 | 312 | 2.43 |
| D-47 | 296 | 2.30 |
| D-48 | 360 | 2.69 |
| D-49 | 326 | 2.69 |
| D-50 | 326 | 2.41 |
| D-51 | 279 | 3.41 |
| D-52 | 279 | 3.37 |
| D-53 | 279 | 3.41 |
| D-54 | 314 | 2.43 |
| D-55 | 322 | 2.64 |
| D-56 | 312 | 2.61 |
| D-57 | 344 | 2.36 |
| D-58 | 346 | 2.28 |
| D-59 | 348 | 2.48 |
| D-60 | 411 | 2.60 |
| D-61 | 336 | 3.29 |
| D-62 | 297 | 3.07 |
| D-63 | 286 | 3.22 |
| D-64 | 320 | 3.16 |
| D-65 | 340 | 3.34 |
| D-66 | 272 | 3.11 |
| D-67 | 300 | 3.31 |
| D-68 | 332 | 3.11 |
| D-69 | 286 | 3.21 |
| D-70 | 316 | 3.19 |
| D-71 | 308 | 3.17 |
| D-72 | 302 | 3.09 |
| D-73 | 324 | 3.26 |
| D-74 | 290 | 3.16 |
| D-75 | 340 | 3.31 |
| D-76 | 300 | 3.27 |
| D-77 | 332 | 3.11 |
| D-78 | 314 | 3.01 |
| D-79 | 340 | 3.31 |
| D-80 | 290 | 3.12 |
| D-81 | 314 | 3.01 |
| D-82 | 300 | 3.24 |
| D-83 | 340 | 3.29 |
| D-84 | 332 | 3.02 |
| D-85 | 316 | 3.21 |
| D-86 | 308 | 3.16 |
| D-87 | 302 | 2.89 |
| D-88 | 358 | 3.27 |
| D-89 | 300 | 3.24 |
| D-90 | 288 | 2.91 |
| D-91 | 356 | 3.31 |
| D-92 | 330 | 3.21 |
| D-93 | 320 | 3.09 |
| D-94 | 304 | 3.21 |
| D-95 | 356 | 3.29 |
| D-96 | 316 | 3.17 |
| D-97 | 378 | 3.36 |
| D-98 | 316 | 3.21 |
| D-99 | 288 | 2.92 |
| D-100 | 320 | 3.11 |
| D-101 | 348 | 3.32 |
| D-102 | 350 | 2.92 |
| D-103 | 288 | 2.91 |
| D-104 | 300 | 3.27 |
| D-105 | 362 | 3.04 |
| D-106 | 304 | 3.21 |
| D-107 | 330 | 3.29 |
| D-108 | 306 | 3.21 |
| D-109 | 364 | 2.99 |
| D-110 | 297 | 3.04 |
| D-111 | 306 | 3.22 |
| D-112 | 366 | 3.14 |
| D-113 | 300 | 3.24 |
| D-114 | 302 | 3.12 |
| D-115 | 302 | 3.12 |
| D-116 | 338 | 3.12 |
| D-117 | 315 | 3.50 |
| D-118 | 288 | 3.08 |
| D-119 | 312 | 3.75 |
| D-120 | 298 | 3.38 |
| D-121 | 269 | 2.63 |
| D-122 | 304 | 3.21 |
| D-123 | 304 | 3.21 |
| D-124 | 293 | 3.06 |
| D-125 | 338 | 2.66 |

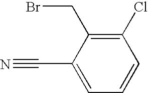

### Intermediate 14. 2-bromomethyl-3-chloro-benzonitrile

2-methyl-3-chloro-benzonitrile (2.00 g, 13.2 mmol) and freshly recrystallized N-bromosuccinimide (2.58 g, 14.5 mmol) were dissolved in carbon tetrachloride (5 mL) and treated with benzoyl peroxide (0.032 g, 0.13 mmol). The reaction mixture was purged under vacuum several times with nitrogen and heated to 85° C. After 16 hours, the mixture was cooled, treated with benzoyl peroxide (30 mg, 0.12 mmol), vacuum purged several times with nitrogen and reheated to 85° C. After 16 hours, the reaction mixture was cooled, stirred with hexanes and filtered. The filter cake was washed with ether and the combined filtrates were concentrated. The crude product was purified by flash chromatography on silica gel (90:10 hexanes:EtOAc to 80:20 hexanes:EtOAc) to afford 2.69 g (88%) of the desired product: ¹H NMR (400 MHz, CDCl₃) δ ppm 4.77 (s, 2H) 7.39 (t, J=7.99 Hz, 1H) 7.62 (dd, J=7.80, 1.36 Hz, 1H) 7.66 (dd, J=8.09, 1.27 Hz, 1H).

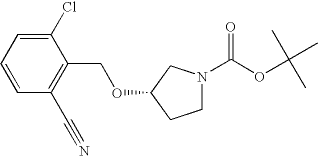

### Intermediate 15. (S)-3-(2-Chloro-6-cyano-benzyloxy)-pyrrolidine-1-carboxylic acid tert-butyl ester

N-(tert-butoxycarbonyl)-(S)-(+)-3-pyrrolidinol (2.40 g, 12.8 mmol) was dissolved in DMF (20 mL) and treated slowly (in small portions) with sodium hydride (0.513 g, 60% dispersion in oil, 12.8 mmol). The reaction mixture was stirred for 30 minutes and 2-bromomethyl-3-chloro-benzonitrile (2.69 g, 11.7 mmol) in DMF (5 mL) was added and washed in with an additional portion of DMF (5 mL). The reaction mixture was stirred at ambient temperature for 16 hours and then concentrated. The residue was dissolved in EtOAc and solids were filtered off. The filtrate was concentrated and purified by flash chromatography on silica gel (90:10 hexanes:EtOAc to 60:40 hexanes:EtOAc) to afford 1.75 g (45%) of the desired product: ¹H NMR (400 MHz, CDCl₃) δ ppm 1.46 (s, 9H) 1.90-2.08 (m, 1H) 2.09-2.24 (m, 1H) 3.36-3.67 (m, 4H) 4.18-4.31 (m, 1H) 4.67-4.87 (m, 2H) 7.39 (t, J=7.99 Hz, 1H) 7.63 (t, J=7.80 Hz, 2H).

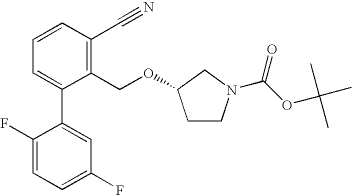

### Intermediate 16. (S)-3-(3-Cyano-2′,5′-difluoro-biphenyl-2-ylmethoxy)-pyrrolidine-1-carboxylic acid tert-butyl ester

(S)-3-(2-Chloro-6-cyano-benzyloxy)-pyrrolidine-1-carboxylic acid tert-butyl ester (0.311 g, 0.923 mmol), 2,5-difluorophenylboronic acid (0.292 g, 1.85 mmol), potassium fluoride (0.161 g, 2.77 mmol), palladium acetate (0.021 g, 0.092 mmol) and dicyclohexylphosphinobiphenyl (0.065 g, 0.19 mmol) were combined, dissolved in THF (3 mL, previously degassed by bubbling with nitrogen for >30 min) and heated to 70° C. After stirring for 16 hours, the reaction mixture was treated with 5% aqueous NaOH and stirred for 16 hours. The biphasic mixture was partitioned between water and dichloromethane. The organic layer was separated and the aqueous was extracted again with dichloromethane. The combined organic layers were dried over sodium sulfate, filtered and concentrated. The residue was purified by flash chromatography on silica gel (90:10 hexanes:EtOAc to 65:35 hexanes:EtOAc) to afford 0.326 g (85%) of desired product: ¹H NMR (400 MHz, CDCl₃) δ ppm 1.45 (s, 9H) 1.81-2.05 (m, 2H) 3.24-3.49 (m, 4H) 4.03-4.09 (m, 1H) 4.38-4.61 (m, 2H) 7.02-7.20 (m, 3H) 7.47-7.56 (m, 2H) 7.76 (dd, J=6.82, 2.34 Hz, 1H).

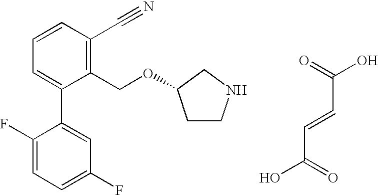

### Example E-1

### (S)-2′,5′-Difluoro-2-(pyrrolidin-3-yloxymethyl)-biphenyl-3-carbonitrile fumarate

(S)-3-(3-Cyano-2′,5′-difluoro-biphenyl-2-ylmethoxy)-pyrrolidine-1-carboxylic acid tert-butyl ester (0.320 g, 0.772 mmol) was dissolved in dichloromethane (4 mL), treated with HCl in ether (1.93 mL, 2.00 M, 3.86 mmol). The reaction mixture was capped and stirred for 16 hours. 5% aqueous NaOH was added and the mixture was partitioned between water and dichloromethane. The organic layer was separated and the aqueous was extracted again with dichloromethane. The combined organic layers were dried over sodium sulfate, filtered and concentrated to 0.225 g of free base. The free base was redissolved in acetone (3 mL) and treated with fumaric acid (83 mg) in acetone (11.9 mL). The mixture was stirred for 16 hours and a white precipitate was filtered, washed with ether and dried under vacuum to afford 0.226 mg (69%) of the desired fumarate salt.

Examples E-2 and E-3 were synthesized in a manner similar to Example E-1.

The names of the compounds of Examples E-2 and E-3 are set out in Table 9. The ¹H NMR and MS values for compounds of Examples E-2 and E-3 are set out in Table 10.

TABLE 9

| EX. | STRUCTURE NAME |
|---|---|
| E-2 | (S)-2′,4′-Difluoro-2-(pyrrolidin-3-yloxymethyl)-biphenyl-3- |
| | carbonitrile fumarate |
| E-3 | (S)-2′,3′-Difluoro-2-(pyrrolidin-3-yloxymethyl)-biphenyl-3- |
| | carbonitrile fumarate |

TABLE 10

| | | MS |
|---|---|---|
| | ¹H NMR | (APCI: |
| Ex. | ((400 MHz, METHANOL-d4) δ ppm) | M + 1) |
| E-1 | 2.0 (dddd, J = 14.3, 9.7, 4.5 Hz, 1H) 2.2 (m, 1H) 3.3 (m, 4H) 4.2 (m, 1H) | 315 |
| | 4.5 (m, 2H) 6.7 (s, 2H) 7.2 (ddd, J = 8.5, 5.6, 3.0 Hz, 1H) 7.3 (m, 2H) | |
| | 7.6 (m, 2H) 7.9 (ddd, J = 9.4, 4.3, 4.0 Hz, 1H) | |
| E-2 | 1.97 (dddd, J = 14.33, 9.84, 9.65, 4.58 Hz, 1H) 2.09-2.25 (m, 1H) | 315 |
| | 3.22-3.37 (m, 4H) 4.19-4.25 (m, 1H) 4.41-4.65 (m, 2H) 6.67 (s, 2H) | |
| | 7.07-7.17 (m, 2H) 7.35-7.42 (m, 1H) 7.57-7.63 (m, 2H) | |
| | 7.85 (dd, J = 5.46, 3.51 Hz, 1H) | |
| E-3 | 1.96 (dddd, J = 14.16, 9.75, 9.63, 4.68 Hz, 1H) 2.05-2.21 (m, 1H) | 315 |
| | 3.21-3.37 (m, 4H) 4.19-4.23 (m, 1H) 4.41-4.67 (m, 2H) 6.64 (s, 1H) | |
| | 7.15 (ddt, J = 7.75, 6.19, 1.61 Hz, 1H) 7.29 (ddt, J = 8.02, 5.02, 1.56 Hz, | |
| | 1H) 7.35-7.44 (m, 1H) 7.58-7.66 (m, 2H) 7.84-7.91 (m, 1H) | |

### Intermediate 17. (S)-3-[(Biphenyl-2-ylmethyl)-amino]-pyrrolidine-1-carboxylic acid tert-butyl ester

To a 0° C. stirred suspension of (S)-3-amino-1-N-BOC-pyrrolidine (1.00 g, 5.37 mmol), biphenyl-2-carbaldehyde (0.978 g, 5.37 mmol) and MgSO₄ (0.646 g, 5.37 mmol) in dry CH₂Cl₂ (53.6 mL) was added sodium triacetoxyborohydride (1.59 g, 7.52 mmol). The reaction was allowed to warm to room temperature overnight with good stirring. The reaction was complete by LC/MS, therefore, the reaction was quenched with saturated NaHCO₃ and the layers were separated. The organic phase was washed twice with 20 ml of brine, dried over MgSO₄, filtered and concentrated under reduced pressure. The residue was purified by silica chromatography (Biotage 40M, Uppsala, Sweden) using a gradient of hexanes:EtOAc (100:0) to hexanes:EtOAc (60:40) to yield 1.22 g (64.5%) of a colorless oil. ¹H NMR (400 MHz, CHLOROFORM-d) δ ppm 1.40 (s, 9H) 1.44-1.53 (m, 1H) 1.80 (dd, J=12.59, 6.15 Hz, 1H) 2.80-2.97 (m, 1H) 3.06-3.15 (m, 1H) 3.18-3.39 (m, 3H) 3.70 (s, 2H) 7.25-7.42 (m, 9H). MS: M+1 (353) Da.

### Intermediate 18. (S)-3-(Biphenyl-2-ylmethyl-ethyl-amino)-pyrrolidine-1-carboxylic-acid tert-butyl ester

To a stirred solution of 3-[(biphenyl-2-ylmethyl)-amino]-pyrrolidine-1-carboxylic acid tert-butyl ester (0.221 g, 0.626 mmol) in dry CH₃CN (6.26 mL) was added Cs₂CO₃ (0.408 g, 1.25 mmol) followed by iodoethane (0.100 mL, 1.25 mmol). The reaction was heated to 70° C. overnight with stirring. HPLC analysis showed some starting material, however, the heat was turned off and the reaction was allowed to cool to room temperature. The mixture was filtered and then concentrated down under reduced pressure. The solid was partitioned between EtOAc (20 mL) and brine (20 mL). The layers were separated, the organic layer was dried over MgSO₄, filtered, and concentrated down under reduced pressure. The oil was purified by silica chromatography (Biotage 40S, Uppsala, Sweden) using a gradient of hexanes:EtOAc (100:0) to hexanes:EtOAc (85:15) to yield 0.157 g (66.1%) of a colorless oil. ¹H NMR (400 MHz, CHLOROFORM-d) δ ppm 0.72-0.84 (m, 3H) 1.40 (s, 9H) 1.56-1.63 (m, 1H) 1.76 (s, 1H) 2.44 (q, J=7.03 Hz, 2H) 2.83-3.03 (m, 1H) 3.07-3.17 (m, 2H) 3.27-3.51 (m, 3H) 3.52-3.59 (m, 1H) 7.16 (d, J=7.03 Hz, 1H) 7.21-7.27 (m, 4H) 7.28-7.40 (m, 4H) 7.61 (d, J=7.81 Hz, 1H). MS: M+1 (381) Da.

### Example F-1

### (S)-Biphenyl-2-ylmethyl-ethyl-pyrrolidin-3-yl-amine fumarate

To a room temperature stirred solution of 3-(biphenyl-2-ylmethyl-ethyl-amino)-pyrrolidine-1-carboxylic-acid tert-butyl ester (0.157 g, 0.414 mmol) in dry EtOAc (1.38 mL) was added an HCl solution in dioxane (1.34 mL, 4.0 M) and the reaction was allowed to stir over night at room temperature. The reaction was complete by HPLC, and was concentrated down under reduced pressure. To a solution of the oil in MeOH (3.2 mL) was added Dowex 550A (OH) anion resin (Sigma-Aldrich Corp., St. Louis, Mo., USA). The mixture was placed on a rotary evaporator for stirring without reduced pressure for 30 minutes. The mixture was filtered, the resin was washed twice with 15 mL of MeOH and concentrated under reduced pressure to yield 0.074 g of the free base. To a solution of the free amine in dry acetone (5 mL) was added fumaric acid (0.031 g, 1.0 eq) and the solution was stirred overnight at room temperature. The suspension was filtered and the solid was washed three times with 15 ml of diethyl ether and the white solid was placed in a drying oven under reduced pressure at 60° C. to yield 0.067 g of the title product. ¹H NMR (400 MHz, METHANOL-d₄) δ ppm 0.82 (t, J=7.02 Hz, 3H) 1.68-1.80 (m, 1H) 1.88-1.97 (m, 1H) 2.49 (q, J=7.08 Hz, 2H) 2.83 (dd, J=11.79, 8.09 Hz, 1H) 3.07-3.18 (m, 2H) 3.22-3.28 (m, 1H) 3.34-3.42 (m, 1H) 3.53-3.71 (m, 2H) 6.68 (s, 2H) 7.18 (d, J=7.41 Hz, 1H) 7.26-7.30 (m, 2H) 7.30-7.38 (m, 3H) 7.41 (t, J=7.12 Hz, 2H) 7.59 (s, 1H). MS: M+1 (280) Da.

### Biological Example 1

Compounds of the present invention (e.g., compounds of Formula I and pharmaceutically acceptable salts thereof) can be assayed for their ability to inhibit a norepinephrine transporter and/or a serotonin transporter. The ability of the compounds of the present invention to inhibit a norepinephrine transporter and/or a serotonin transporter can be determined using conventional radioligand receptor transport assays. For example, the transporters can be heterologously expressed in cell lines and experiments conducted in membrane preparations from the cell lines that express a norepinephrine transporter and/or a serotonin transporter.

The compounds of the Examples listed in Tables 11 to 15 below were tested as follows for NET and SERT binding activity.

hNET Receptor Binding:

Cell pastes of HEK-293 cells transfected with a human norepinephrine transporter cDNA were prepared. The cell pastes were resuspended in 400 to 700 ml of Krebs-HEPES assay buffer (25 mM HEPES, 122 mM NaCl, 3 mM KCl, 1.2 mM MgSO₄, 1.3 mM CaCl₂, and 11 mM glucose, pH 7.4) with a Polytron homogenizer at setting 7 for 30 seconds. Aliquots of membranes (5 mg/ml protein) were stored in liquid nitrogen until used.

The binding assay was set up in Beckman deep-well polypropylene plates with a total volume of 250 μl containing: test compound (10⁻⁵M to 10⁻¹²M), cell membranes, and 50 pM [¹²⁵I]-RTI-55 (Perkin Elmer, NEX-272; specific activity 2200 Ci/mmol). The reaction was incubated by gentle agitation for 90 minutes at room temperature and was terminated by filtration through Whatman GF/C filter plates using a Brandel 96-well plate harvester. Scintillation fluid (100 μl) was added to each well, and bound [¹²⁵I]-RTI-55 was determined using a Wallac Trilux Beta Plate Counter. Test compounds were run in duplicate, and specific binding was defined as the difference between binding in the presence and absence of 10 μM desipramine.

Excel and GraphPad Prism software were used for data calculation and analysis. IC₅₀ values were converted to K, values using the Cheng-Prusoff equation. The Kᵢ values (nM) for the hNET are reported below in Tables 11 to 15.

hSERT Receptor Binding

Cell pastes of HEK-293 cells transfected with a human serotonin transporter cDNA were prepared. The cell pastes were resuspended in 400 to 700 ml of Krebs-HEPES assay buffer (25 mM HEPES, 122 mM NaCl, 3 mM KCl, 1.2 mM MgSO₄, 1.3 mM CaCl₂, and 11 mM glucose, pH 7.4) with a Polytron homogenizer at Setting 7 for 30 seconds. Aliquots of membranes (˜2.5 mg/ml protein) were stored in liquid nitrogen until used.

Assays were set up in FlashPlates pre-coated with 0.1% PEI in a total volume of 250 μl containing: test compound (10⁻⁵M to 10⁻¹²M), cell membranes, and 50 pM [¹²⁵I]-RTI-55 (Perkin Elmer, NEX-272; specific activity 2200 Ci/mmol). The reaction was incubated and gently agitated for 90 minutes at room temperature, and terminated by removal of assay volume. Plates were covered, and bound [¹²⁵I]-RTI-55 was determined using a Wallac Trilux Beta Plate Counter. Test compounds were run in duplicate, and specific binding was defined as the difference between binding in the presence and absence of 10 μM citalopram.

Excel and GraphPad Prism software were used for data calculation and analysis. IC₅₀ values were converted to Kᵢ values using the Cheng-Prusoff equation. The Kᵢ values (nM) for the hSERT are reported below in Tables 11 to 15.

TABLE 11

| | NET Kᵢ | SERT Kᵢ |
|---|---|---|
| Ex. | (nM) | (nM) |
| A-2 | 7.60 | 93.32 |
| A-3 | 10.85 | 133.80 |
| A-1 | 10.91 | 103.00 |
| A-4 | 11.21 | 138.70 |
| A-5 | 11.30 | 11.98 |
| A-6 | 12.89 | 65.57 |
| A-7 | 13.50 | 34.50 |
| A-8 | 15.31 | 20.47 |
| A-9 | 17.70 | 173.58 |
| A-10 | 22.78 | 116.18 |
| A-11 | 39.24 | 10.28 |
| A-12 | 50.03 | 81.05 |
| A-13 | 65.97 | 54.04 |
| A-14 | 67.50 | 204.00 |
| A-15 | 80.94 | 184.90 |
| A-16 | 89.88 | 436.30 |
| A-17 | 93.53 | 15.12 |
| A-18 | 102.60 | 12.98 |
| A-19 | 111.49 | 751.98 |
| A-20 | 127.40 | 312.50 |
| A-21 | 610.24 | 307.46 |
| A-22 | 10,000.00 | 136.47 |
| A-23 | 15.79 | 102.04 |
| A-24 | 14.25 | 232.08 |
| A-25 | 26.21 | 152.43 |
| A-26 | 30.60 | 621.80 |
| A-27 | 2,205.89 | 210.55 |
| A-28 | 4,855.14 | 1,758.08 |
| A-29 | 2,911.61 | 2,469.99 |
| A-30 | 80.15 | 247.35 |
| A-31 | 46.31 | 142.31 |
| A-32 | 23.86 | 757.83 |
| A-33 | 25.7 | 99.67 |
| A-34 | 23.32 | 438.61 |

TABLE 12

| | NET Kᵢ | SERT Kᵢ |
|---|---|---|
| Ex. | (nM) | (nM) |
| B-2 | 206.00 | 3,348.00 |
| B-1 | 1,016.00 | 5,647.00 |
| B-3 | 310.30 | 1,197.00 |

TABLE 13

| | NET Kᵢ | SERT Kᵢ |
|---|---|---|
| Ex. | (nM) | (nM) |
| C-1 | 382.48 | 108.34 |
| C-2 | 845.08 | 74.62 |

TABLE 14

| | NET Kᵢ | SERT Kᵢ |
|---|---|---|
| Ex. | (nM) | (nM) |
| D-1 | 4.84 | 39.17 |
| D-2 | 8.42 | 260.50 |
| D-3 | 14.68 | 304.40 |
| D-4 | 15.10 | 18.30 |
| D-5 | 17.90 | 197.00 |
| D-6 | 18.53 | 18.40 |
| D-7 | 21.29 | 110.10 |
| D-8 | 21.95 | 208.40 |
| D-9 | 23.30 | 71.60 |
| D-10 | 26.52 | 317.20 |
| D-11 | 39.63 | 26.72 |
| D-12 | 41.61 | 100.50 |
| D-13 | 43.79 | 21.63 |
| D-14 | 47.30 | 15.10 |
| D-15 | 55.40 | 272.10 |
| D-16 | 63.47 | 24.72 |
| D-17 | 87.00 | 30.60 |
| D-18 | 102.20 | 217.90 |
| D-19 | 107.80 | 346.00 |
| D-20 | 108.30 | 24.82 |
| D-21 | 112.90 | 431.20 |
| D-22 | 139.50 | 27.29 |
| D-23 | 177.20 | 154.10 |
| D-24 | 229.80 | 1,263.00 |
| D-25 | 268.60 | 124.40 |
| D-26 | 276.80 | 456.90 |
| D-27 | 295.00 | 91.93 |
| D-28 | 301.20 | 1,101.00 |
| D-29 | 313.80 | 171.80 |
| D-30 | 370.50 | 284.50 |
| D-31 | 464.10 | 3,099.00 |
| D-32 | 515.50 | 548.90 |
| D-33 | 525.90 | 28.87 |
| D-34 | 554.40 | 2,748.40 |
| D-35 | 585.20 | 59.63 |
| D-36 | 635.20 | 149.90 |
| D-37 | 1,000.00 | 5,649.30 |
| D-38 | 1,189.00 | 39.31 |
| D-39 | 1,253,00 | 527.60 |
| D-40 | 1,263.00 | 919.30 |
| D-41 | 1,279.00 | 345.40 |
| D-42 | 2,539.00 | 10,000.00 |
| D-43 | 2,666.70 | 16.90 |
| D-44 | 3,340.70 | 12.40 |
| D-45 | 3,340.70 | 12.40 |
| D-46 | 3,880.00 | 3,759.00 |
| D-47 | 4,123.00 | 906.40 |
| D-48 | 4,182.50 | 10,000.00 |
| D-49 | 4,230.40 | 2,967.40 |
| D-50 | 4,449.00 | 709.00 |
| D-51 | 5,410.15 | 5,459.05 |
| D-52 | 10,000.00 | 10,000.00 |
| D-53 | 10,000.00 | 10,000.00 |
| D-54 | 10,000.00 | 10,000.00 |
| D-55 | 10,000.00 | 201.10 |
| D-56 | 10,000.00 | 658.00 |
| D-57 | 10,000.00 | 588.70 |
| D-58 | 10,000.00 | 3,030.90 |
| D-59 | 10,000.00 | 57.80 |
| D-60 | 10,000.00 | 882.00 |
| D-61 | 992.60 | 793.60 |
| D-62 | 479.60 | 318.80 |
| D-63 | 36.60 | 423.20 |
| D-64 | 1,291.00 | 656.30 |
| D-65 | 265.50 | 49.70 |
| D-66 | 65.90 | 125.60 |
| D-67 | 376.90 | 116.00 |
| D-68 | 1,161.90 | 1,237.70 |
| D-69 | 90.40 | 85.80 |
| D-70 | 831.90 | 195.40 |
| D-71 | 60.00 | 107.80 |
| D-72 | 205.10 | 355.60 |
| D-73 | 146.00 | 49.70 |
| D-74 | 43.40 | 146.00 |
| D-75 | 597.50 | 105.80 |
| D-76 | 1,278.80 | 733.60 |
| D-77 | 1,345.20 | 1,401.30 |
| D-78 | 6,872.80 | 444.40 |
| D-79 | 6,783.50 | 206.30 |
| D-80 | 36.00 | 44.70 |
| D-81 | 1,967.90 | 371.00 |
| D-82 | 257.90 | 77.10 |
| D-83 | 108.90 | 71.60 |
| D-84 | 1,916.30 | 70.40 |
| D-85 | 706.00 | 324.40 |
| D-86 | 278.50 | 76.20 |
| D-87 | 3,077.50 | 124.40 |
| D-88 | 1,028.80 | 1,037.30 |
| D-89 | 188.90 | 268.40 |
| D-90 | 68.30 | 516.20 |
| D-91 | 935.30 | 16.50 |
| D-92 | 738.30 | 144.00 |
| D-93 | 1,116.60 | 1,448.90 |
| D-94 | 102.00 | 88.40 |
| D-95 | 330.90 | 122.20 |
| D-96 | 591.10 | 1,384.10 |
| D-97 | 147.80 | 286.10 |
| D-98 | 187.30 | 291.70 |
| D-99 | 43.10 | 658.90 |
| D-100 | 403.50 | 485.80 |
| D-101 | 962.10 | 3,779.90 |
| D-102 | 10,000.00 | 10,000.00 |
| D-103 | 650.20 | 1,086.80 |
| D-104 | 112.20 | 219.90 |
| D-105 | 1,522.60 | 577.30 |
| D-106 | 121.50 | 371.70 |
| D-107 | 3,830.10 | 386.20 |
| D-108 | 102.90 | 57.00 |
| D-109 | 10,000.00 | 10,000.00 |
| D-110 | 1,203.00 | 1,322.00 |
| D-111 | 97.00 | 155.30 |
| D-112 | 9629.96 | 107.29 |
| D-113 | 1,338.60 | 1,470.70 |
| D-114 | 145.00 | 60.00 |
| D-115 | 673.90 | 606.00 |
| D-116 | 688.50 | 38.70 |
| D-117 | 10,000.00 | 2,164.88 |
| D-118 | 10,000.00 | 3,558.49 |
| D-119 | 963.33 | 2,734.61 |
| D-120 | 571.13 | 4,227.43 |
| D-121 | 10,000.00 | 5,627.24 |
| D-122 | 659.09 | 769.20 |
| D-124 | 7,397.76 | 2,734.38 |
| D-125 | 10,000.00 | 6,925.45 |

TABLE 15

| | NET Kᵢ | SERT Kᵢ |
|---|---|---|
| Ex. | (nM) | (nM) |
| E-1 | 336.0 | 1115.9 |
| E-2 | 763.2 | 3768.9 |
| E-3 | 559.8 | 1574.1 |

### Biological Example 2

Compounds of the present invention may be assayed for their ability to alleviate capsaicin-induced mechanical allodynia in a rat (e.g., Sluka (2002) J of Neuroscience, 22(13): 5687-5693). For example, a rat model of capsaicin-induced mechanical allodynia was carried out as follows:

On day 0, male Sprague-Dawley rats (˜150 g) in the dark cycle were placed in suspended wire-bottom cages and allowed to acclimate for 0.5 hour in a darkened, quiet room. The day 0 paw withdrawal threshold (PWT) was determined on the left hind paw by Von Frey hair assessment using the Dixon up and down method. After assessment, the plantar muscle of the right hind paw was injected with 100 μl capsaicin (0.25% (w/v) in 10% ethanol, 10% Tween 80, in sterile saline). On day 6 the PWT of the left hind paw (contralateral from injection site) was determined for each animal. Animals from the day 6 prereads with PWT ≦11.7 g were considered allodynic responders and were regrouped so that each cage had similar mean PWT values. On day 7, responders were dosed subcutaneously with 10 mg compound/kg body weight, or with vehicle alone. The vehicle was phosphate buffered saline containing 2% Cremophor® EL (BASF). The contralateral PWT values were determined at 1 hour after the single dose, with the investigator blinded to the dosing scheme.

For each animal, the day 6 PWT value was subtracted from the 1 hour PWT value to give a delta PWT value that represents the change in PWT due to the 1 hour drug treatment. In addition, the day 6 PWT was subtracted from the day 0 PWT to give the baseline window of allodynia present in each animal. To determine % inhibition of allodynia of each animal normalized for vehicle controls, the following formula was used: % Inhibition of Allodynia=100×[(Delta PWT(drug)−mean Delta PWT(vehicle))/(Baseline−mean Delta PWT(vehicle))].

The mean percent Inhibition of allodynia values (for eight animals assayed for each compound)+/−the standard error of the mean (SEM) are shown in Table 15. Compounds exhibiting a greater than 30% inhibition in allodynia assay are considered active.

TABLE 15

| | % INHIBITION +/− |
|---|---|
| Ex. | SEM |
| A-1 | 40.90 +/− 10.2 |
| A-3 | 17.40 +/− 9.9 |
| A-4 | 30.20 +/− 1.8 |
| A-6 | 66.20 +/− 7.8 |

### Biological Example 3

Several compounds were evaluated for their affinity for the neuronal α-BGTX-insensitive central nicotinic receptor in the rat cerebral cortex determined in a radioligand binding assay.

Experimental protocol Membrane homogenates of cerebral cortex (800 μg protein) are incubated for 75 min at 4° C. with 1.5 nM [³H]cytisine in the absence or presence of the test compound (10 μM in a buffer containing 50 mM Tris-HCl (pH 7.4), 120 mM NaCl, 5 mM KCl, 1 mM MgCl₂ and 2 mM CaCl₂.

Nonspecific binding is determined in the presence of 10 μM nicotine.

Following incubation, the samples are filtered rapidly under vacuum through glass fiber filters (Filtermat B, Wallac) presoaked with 0.3% PEI (polyethyleneimine) and rinsed several times with ice-cold 50 mM Tris-HCl using a 48-sample cell harvester (Mach II, Tomtec). The filters are dried then counted for radioactivity in a scintillation counter (Betaplate 1204, Wallac) using a solid scintillator (Meltilex B/HS, Wallac).

The results are expressed as a percent inhibition of the control radioligand specific binding.

The standard reference compound is nicotine, which is tested in each experiment at several concentrations to obtain a competition curve from which its IC₅₀ is calculated.

The compound of Examples A-18, A-17, and A-7 each did not inhibit more than 21% of control activity at 10 μM as shown in Table 16:

TABLE 16

| | Percent inhibition |
|---|---|
| | at 10 μM of |
| Example | compound |
| A-18 | 21% |
| A-17 | 15% |
| A-7 | 5% |

It is understood that the examples and embodiments described herein are for illustrative purposes only and that various modifications or changes in light thereof will be apparent to persons skilled in the art and are to be included within the spirit and purview of this application and the scope of the appended claims. All publications, patents, and patent applications cited herein are hereby incorporated by reference in their entirety for all purposes.

What is claimed is:

1. A compound of Formula I 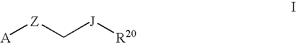
or a pharmaceutically acceptable salt thereof, wherein:
R²⁰ is a 3-pyrrolidinyl optionally substituted with one to eight substituents each independently selected from the group consisting of: C₁-C₄ alkyl, and halo;
J is O or —N—R²², wherein R²² is H, C₁-C₄ alkyl, or —C(O)—C₁-C₄alkyl;
Z is selected from the group consisting of: phenylene, naphthylene, a 5 to 6 membered heteroarylene, a 9 to 11-membered bicyclic arylene, and a 8 to 10-membered bicyclic heteroarylene, any of which may be optionally substituted with 1 to 5 substituents independently selected from the group consisting of: C₁-C₄ alkyl, C₁-C₄ alkoxy, halo, —CF₃, —CN, OH, C₁-C₄ alkyl-S—, and —NR³⁰R³¹,
R³⁰ and R³¹ are each independently selected from the group consisting of: H, and C₁-C₄ alkyl;
A is selected from the group consisting of: phenyl, naphthyl, a 5 to 6 membered heteroaryl, a 9 to 11-membered bicyclic aryl, and a 8 to 10-membered bicyclic heteroaryl, any of which may be optionally substituted with 1 to 5 substituents independently selected from the group consisting of: C₁-C₄ alkyl, C₁-C₄ alkoxy, halo, OH, —CN, —CF₃, CF₃O—, C₁-C₄ alkyl-S—, phenyl, C₁-C₄ alkyl-C(O)—, a 5 or 6 membered heteroaryl, a 5 to 7-membered heterocycloalkyl, C₁-C₄ alkyl-sulfonyl, —C(O)O—R¹², —C(O)NR¹⁴R¹⁶, —NR¹⁰R¹¹, —O-phenyl, and —O—CH₂-phenyl:
R¹⁰ and R¹¹ are each independently selected from the group consisting of: H, —C(O)—C₁-C₄ alkyl, and C₁-C₄ alkyl;
R¹² is H or C₁-C₄ alkyl; and
R¹⁴ and R¹⁶ are each independently selected from the group consisting of: H and C₁-C₄ alkyl.

2. The compound of claim 1, or a pharmaceutically acceptable salt thereof, wherein said compound is of formula II 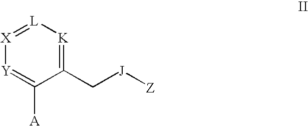
wherein:
K is CR¹ or N;
L is CR² or N;
X is CR³ or N;
Y is CR⁴ or N;
where zero, one, or two of K, L, X and Y are N;
R¹, R², R³, and R⁴ are each independently selected from the group consisting of:
H, C₁-C₄ alkyl, C₁-C₄ alkoxy, halo, —CF₃, —CN, OH, C₁-C₄ alkyl-S—, and —NR³⁰R³¹, wherein R³⁰ and R³¹ are each independently selected from the group consisting of: H, and C₁-C₄ alkyl.

3. The compound of claim 2, or a pharmaceutically acceptable salt thereof, wherein A is 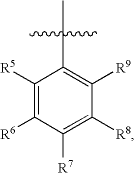
R⁵, R⁶, R⁷, R⁸, and R⁹ are each independently selected from the group consisting of: H, C₁-C₄ alkyl, C₁-C₄ alkoxy, halo, OH, —CN, —CF₃, CF₃O—, C₁-C₄ alkyl-S—, phenyl, C₁-C₄ alkyl-C(O)—, a 5 or 6 membered heteroaryl, a 5 to 7-membered heterocycloalkyl, C₁-C₄ alkyl-sulfonyl, —C(O)O—R¹², —C(O)NR¹⁴R¹⁶, NR¹⁰R¹¹, and —O—CH₂-phenyl,
R¹⁰ and R¹¹ are each independently selected from the group consisting of: H, C(O)—C₁-C₄ alkyl, and C₁-C₄ alkyl;
R¹² is H, or C₁-C₄ alkyl; and
R¹⁴ and R¹⁶ are each independently selected from the group consisting of: H and C₁-C₄ alkyl.

4. The compound of claim 3, or a pharmaceutically acceptable salt thereof, wherein the carbon of the 3-position of said optionally substituted 3-pyrrolidinyl has the S configuration.

5. The compound of claim 3, or a pharmaceutically acceptable salt thereof, wherein J is O; K is CR¹; L is CR²; X is CR³; Y is CR⁴; and R²⁰ is an unsubstituted pyrrolidinyl.

6. The compound of claim 5, or a pharmaceutically acceptable salt thereof, wherein R⁵, R⁶, R⁷, R⁸, and R⁹ are each independently selected from the group consisting of: H, C₁-C₄ alkyl, C₁-C₄ alkoxy, and halo.

7. The compound of claim 1, wherein said compound is (S)-3-(2′,4′-difluoro-biphenyl-2-ylmethoxy)-pyrrolidine hydrochloride, or said compound is selected from the group consisting of:
(S)-3-(4,2′,4′-trifluoro-biphenyl-2-ylmethoxy)-pyrrolidine;
(S)-3-(4,2′,3′-trifluoro-biphenyl-2-ylmethoxy)-pyrrolidine;
(S)-3-(4,2′-difluoro-biphenyl-2-ylmethoxy)-pyrrolidine;
(S)-3-(4′-methyl-biphenyl-2-ylmethoxy)-pyrrolidine;
(S)-3-(3′-4′-difluoro-biphenyl-2-ylmethyloxy)-pyrrolidine;
(S)-3-(2′,3′-difluoro-biphenyl-2-ylmethoxy)-pyrrolidine;
(S)-3-(biphenyl-2-ylmethoxy)-pyrrolidine;
(S)-3-(4′-fluoro-biphenyl-2-ylmethoxy)-pyrrolidine; and
(S)-3-(2′,4′-difluoro-biphenyl-2-ylmethoxy)-pyrrolidine;
or a pharmaceutically acceptable salt thereof.

8. A method of treating fibromyalgia, the method comprising administering to a mammal in need of such treatment a therapeutically effective amount of a compound of claim 1, or a pharmaceutically acceptable salt thereof.

9. A method of treating a disease selected from the group consisting of: attention deficit hyperactivity disorder, neuropathic pain, urinary incontinence, generalized anxiety disorder, depression and schizophrenia, the method comprising administering to a mammal in need of such treatment a therapeutically effective amount of a compound of claim 1, or a pharmaceutically acceptable salt thereof.

10. A pharmaceutical composition comprising: a therapeutically effective amount of a compound of claim 1, or pharmaceutically acceptable salt thereof, and a pharmaceutically acceptable excipient.
